(19) United States
(12) Patent Application Publication
(10) Pub. No.: US 2016/0263140 A1
(43) Pub. Date: Sep. 15, 2016

(54) USE OF COMPOUNDS ACTIVATING SIRT-3 FOR MIMICKING EXERCISE

(71) Applicant: President and Fellows of Harvard College, Cambridge, MA (US)

(72) Inventors: David A. Sinclair, Chestnut Hill, MA (US); Juan Carmona, Belton, TX (US)

(21) Appl. No.: 14/884,681

(22) Filed: Oct. 15, 2015

Related U.S. Application Data
(63) Continuation of application No. 12/739,428, filed on Dec. 15, 2010, now abandoned.
(60) Provisional application No. 60/981,960, filed on Oct. 23, 2007.

(51) Int. Cl. A61K 31/7064

(52) U.S. Cl. CPC A61K 31/7064

(57) ABSTRACT

The invention relates to modulation of SIRT3 activity levels. The invention has applications for regulating metabolism and mimicking caloric restriction or exercise in a muscle cell.

### RELATED APPLICATIONS

This application is a continuation of U.S. application Ser. No. 12/739,428, filed Dec. 15, 2010, entitled “USE OF COMPOUNDS ACTIVATING SIRT-3 FOR MIMICKING EXERCISE,” which is a national stage filing under 35 U.S.C. §371 of international application PCT/US2008/012058, filed Oct. 23, 2008, which was published under PCT Article 21(2) in English, and claims the benefit under 35 U.S.C. §119(e) of U.S. Provisional Application Ser. No. 60/981,960, entitled “SIRT-3 Related Methods and Compositions for Mimicking Exercise,” filed on Oct. 23, 2007, the disclosures of which are herein incorporated by reference in their entireties.

### FEDERALLY SPONSORED RESEARCH

This invention was made with Government support under grants RO1GM068072, RO1AG028730 and PO1AG027916 awarded by the National Institutes of Health. The Government has certain rights to this invention.

### FIELD OF THE INVENTION

The invention relates to methods of mimicking caloric restriction and increasing metabolism by modulating SIRT3 activity.

### BACKGROUND OF INVENTION

The sirtuins are a conserved family of deacetylases and mono-ADP-ribosyltransferases that use NAD⁺ as a co-substrate (Guarente and Picard, 2005). These unusual enzymes, which bear virtually no sequence homology to Class I and II HDACs (Denu. 2005; Frye, 2000), have emerged as key regulators of cell survival and organismal longevity (Guarente and Picard. 2005). The founding member of the sirtuin family, Saccharomyces cerevisiae Sir2, is an NAD⁺-dependent histone deacetylase that mediates lifespan extension by mild stress and calorie restriction (CR) (Imai et al., 2000; Lin et al., 2000; Rogina and Helfand, 2004; Smith et al., 2000; Tanny et al., 1999). Mammals have seven sirtuins, SIRT1-7. SIRT1, a nuclear deacetylase, regulates a variety of functions including cell survival, glucose homeostasis, and fat metabolism (Guarente, 2005). There are three mitochondrial sirtuins, SIRT3-5. SIRT3 and SIRT4 were recently shown to regulate acetyl-CoA synthetase 2 (AceCS2) and glutamate dehydrogenase, respectively (Haigis et al., 2006; Hallows et al., 2006; Schwer et al., 2002), but little else is known about their biological functions.

### SUMMARY OF INVENTION

Aspects of the invention relate to modulation of SIRT3 expression and activity. SIRT3 is demonstrated herein to be expressed at high levels in metabolically active tissue. Modulation of SIRT3 has a variety of physiological applications for muscle cells including mimicking calorie restriction or exercise, increasing mitochodrial biogenesis or metabolism, sensitizing a cell to glucose uptake, increasing fatty acid oxidation, and decreasing reactive oxygen species. In addition, SIRT3 is demonstrated herein to be involved in promoting cell survival during genotoxic stress. Thus modulation of SIRT3 levels also has applications in mediating cell survival.

Aspects of the invention relate to methods for mimicking the benefits of calorie restriction or exercise in a muscle cell by contacting a muscle cell with an agent that increases the protein or activity level of SIRT3 in the cell. In some embodiments, the invention relates to methods for increasing mitochondrial biogenesis or metabolism or for boosting mitochondrial activity/endurance in a muscle cell by contacting a muscle cell with an agent that increases the protein or activity level of SIRT3 in the cell. In some embodiments, the invention relates to methods for sensitizing a muscle cell to glucose uptake by contacting a muscle cell with an agent that increases the protein or activity level of SIRT3 in the cell.

Further embodiments of the invention relate to methods for increasing fatty acid oxidation in a muscle cell by contacting a muscle cell with an agent that increases the protein or activity level of SIRT3 in the cell. Some embodiments of the invention relate to methods for decreasing reactive oxygen species (ROS) in a muscle cell by contacting the muscle cell with an agent that increases the protein or activity level of SIRT3 in the cell.

Described herein are methods for increasing PGC-1α and/or ucp3 and/or GLUT4 expression and/or for activating AMP activated protein kinase (AMPK) in a muscle cell by contacting a muscle cell with an agent that increases the protein or activity level of SIRT3 in the cell. In some embodiments, methods described herein relate to muscle cells such as skeletal muscle cells. In certain embodiments, the muscle cell is a cell of a slow-twitch muscle. In some embodiments, the muscle cell is a soleus muscle cell. In certain embodiments, the muscle cell is in a subject and the method involves administering to the subject, who is a subject in need thereof, the agent that increases the protein or activity level of SIRT3 in cells of the subject.

Aspects of the invention relate to targeting or administering agents into a muscle of the subject in need thereof. In some embodiments the agent is a SIRT3 protein or biologically effective homolog thereof or a nucleic acid encoding such. In certain embodiments, the agent is a nucleic acid encoding human SIRT3 or a biologically effective homolog thereof. In some embodiments, the biologically effective homolog is a fragment of human SIRT3 having deacetylase and/or ADP ribosyltransferase activity. The agent can be a small molecule and in some embodiments is an activator of SIRT1. Methods described herein can be applied to a cell in need of SIRT3 increase in protein or activity level. In some embodiments, the cell is a diseased cell of a subject.

Aspects of the invention relate to methods for regulating skeletal muscle metabolism or skeletal muscle energy homeostasis in a subject by administering to a subject in need thereof an agent that modulates the protein or activity level of SIRT3 in the subject. Some embodiments of the invention relate to methods for regulating skeletal muscle metabolism or skeletal muscle energy homeostasis in a subject by administering to a subject in need thereof an agent that modulates the protein or activity level of AMPK. In some embodiments, methods relate to increasing skeletal muscle metabolism or skeletal muscle energy homeostasis in a subject, comprising administering to the subject an agent that stimulates the protein or activity level of AMPK.

Described herein are methods for increasing the protein level of SIRT3 in a muscle cell by subjecting the cell to caloric restriction or fasting. In some embodiments, the method relates to increasing the protein level of SIRT3 in the muscles of a subject, comprising subjecting the subject to caloric restriction, fasting or exercise. Aspects of the invention relate to methods for treating or preventing a disease or condition in a subject in which muscle cells would benefit from calorie restriction or exercise, by administering to a subject in need thereof an agent that increases the protein or activity level of SIRT3 in a muscle cell. In some embodiments, the disease or condition is a mitochondrial disease, a metabolic disorder, a neurologic disorder, a muscular disorder, a cardiovascular disease, excessive weight or obesity. In certain embodiments, the disease is insulin resistance or diabetes or a diabetes related condition or disorder. In some embodiments, the disease is metabolic syndrome.

Further aspects of the invention relate to methods for determining whether a subject has or is likely to develop a disease or condition that is associated with a defect in muscle metabolism or muscle energy homeostasis, by determining the level of protein or activity of SIRT3 in a muscle cell of the subject, wherein a statistically lower level of protein or activity of SIRT3 in the cell of the subject, relative to a control, indicates that the subject has or is likely to develop a disease or condition that is associated with a defect in muscle metabolism or muscle energy homeostasis. In some embodiments, the control is the level of protein or activity of SIRT3 in a similar muscle cell of a healthy subject, or that of an average of at least healthy subjects.

Also described herein are methods for identifying an agent that mimics the benefits of calorie restriction or exercise in a muscle cell, comprising contacting a muscle cell with or administering into a muscle cell an agent; and determining whether the benefits of calorie restriction or exercise are mimicked in the cell. In some embodiments, the agent increases the protein or activity level of SIRT3 in a muscle cell. In some embodiments, the method further involves determining whether the agent increases the protein or activity level of SIRT3 in a muscle cell. In certain embodiments, the agent increases the protein or activity level of SIRT3 and SIRT1 in a muscle cell. The method can further involve determining whether the agent increases the protein or activity level of SIRT3 and SIRT1 in a muscle cell. In some embodiments the cell is a eukaryotic cell. In certain embodiments the cell is a mammalian cell, such as a human cell.

### BRIEF DESCRIPTION OF DRAWINGS

The accompanying drawings are not intended to be drawn to scale. For purposes of clarity, not every component may be labeled in every drawing. In the drawings:

FIGS. 1A-1E depict graphs and Western blots indicating skeletal muscle-specific induction of SIRT3 and associated downstream markers in exercise-trained mice. FIG. 1A shows Western blots indicating SIRT3 expression in Cardiac tissue and triceps muscle. Triceps or cardiac muscle tissue was homogenized and 50 μg of protein was analyzed by Western blot, using anti-SIRT3 serum (Covance) and α-tubulin control; representative blots are shown here and throughout. SED=sedentary and TRD=trained. FIG. 1B shows quantification of SIRT3 band intensities using ImageQuant from blots with animals grouped by gender. Males are plotted as open bars and females are shaded bars. Total number of animals used per cohort and graphed is as follows: sedentary males, N=7; sedentary females, N=5; exercised males, N=8; exercised females, N=6. FIG. 1C shows Western blot analysis of Phospho-CREB and total CREB protein expression using 50 μg of triceps protein lysate from trained vs. sedentary mice. Band intensities of phospho-CREB/Ser133 and CREB were quantified and phospho-CREB content was normalized relative to total CREB content; insert provides sample blots of male triceps tissue. FIG. 1D presents a Western blot and a graph showing that induction of PGC-1α correlates with enhanced SIRT3 expression in triceps; samples processed and analyzed as above; insert blots are of male triceps tissue. FIG. 1E presents a graph of citrate synthase activity which was measured as a mitochondrial marker from the same triceps samples, as described previously (52). N=2, *P<0.05, **P<0.01.

FIG. 2 is a schematic diagram indicating SIRT3 action in the skeletal myocyte. Collectively, the data supports a working model in which SIRT3 responds dynamically to various nutritional and physiological signals to impact muscle energy homeostasis via AMPK and downstream processes, such as β-oxidation, ROS production, mitochondrial biogenesis, and glucose uptake. Given this role, therefore, SIRT3 action within the skeletal muscle cells may serve as an important diagnostic and therapeutic target for impacting human health and disease.

FIGS. 3A-3H present Western blots and graphs indicating that Nampt is a stress- and nutrient-responsive gene that protects cells against the genotoxic agent MMS. FIGS. 3A-3B present Western blots indicating Nampt levels in human fibrosarcoma HT1080 cells in the presence or absence of 10% FBS (FIG. 3A) or of liver tissue extracts from fed or 2 day-fasted Sprague-Dawley rats (FIG. 3B). Actin and tubulin were used as loading controls. FIGS. 3C-3D present graphs indicating Nampt protein (FIG. 3C) and mRNA levels (FIG. 3D) in livers of fasted rats (n=4; bars represent the mean of three experiments±s.d. using student t-test). FIG. 3E presents a Western blot of Nampt in primary rat cardiomyocytes under hypoxia and/or serum starvation. FIGS. 3F-3G present Western blots and graphs indicating survival of HT1080 cells stably expressing human (FIG. 3F) or transiently expressing mouse Nampt (FIG. 3G) following treatment with 1.2 mM methylmethanesulfonate (MMS). FIG. 3H presents a Western blot and a graph indicating survival of human kidney HEK293 cells stably expressing human Nampt treated with MMS as in FIG. 3F.

FIGS. 4A-4F present Western blots and graphs indicating that Nampt protects against apoptotic cell death induced by topoisomerase inhibitors. FIG. 4A presents a Western blot and a graph indicating sensitivity of HT1080 with siRNA-mediated knockdown of NAMPT after MMS exposure. FIG. 4B presents a graph indicating that stable overexpression of Nampt enhances survival of HEK293 cells following MMS treatment and the effect is blocked by the Nampt-inhibitor FK866. FIG. 4C presents graphs indicating survival of WT or Nampt knockdown HT1080 cells after serum deprivation for 22 h and then exposed to MMS for 17 h. Serum deprivation upregulates Nampt and enhances survival of WT but not Nampt knockdown HT1080 cells. FIGS. 4D-4E present Western blots and graphs indicating survival of HEK293 stably-overexpressing Nampt (FIG. 4D) or HT1080 with siRNA knockdown of Nampt (FIG. 4E) following etoposide treatment. FIG. 4F shows a Western blot and a graph indicating survival of HT1080 Nampt knockdown cells after camptothecin treatment. Apoptosis was assessed by Western blot analysis of cleaved Caspase-3. Bars represent the mean of three experiments±s.d.

FIGS. 5A-5G present Western blots and graphs indicating that Nampt-mediated protection against genotoxicity requires SIRT3 and SIRT4. FIG. 5A presents a graph showing survival of HEK293 cells stably expressing Nampt following exposure to MMS in the presence or absence of the SIRT1-specific inhibitor EX-527. FIG. 5B presents a graph showing siRNA knockdown of SIRT1 using a pool of four siRNA oligos, compared to non-targeting siRNA controls. Cells were co-transfected with FAM-tagged fluorescent oligos and percentage cell death was determined by FACS as a ratio of PI/FAM positive cells versus total FAM positive cells. FIG. 5C presents a graph showing survival of HEK293 cells that were treated with 100 μM sirtinol, a pan sirtuin inhibitor. All experiments were carried out three times in triplicate. Bars represent the mean of three experiments±s.d. FIGS. 5D-5E present graphs indicating cell survival. SIRT3 or SIRT4 were knocked down in HEK293 cells stably-overexpressing Nampt using pools of specific siRNA oligos and cells were then treated with MMS and scored for survival. FIG. 5F shows a Western blot wherein cells from (FIG. 5D) were probed for cleaved caspase-3, an indicator of apoptosis. FIG. 5G presents a Western blot showing the levels of acetylated AceCS2 in immunoprecipitation (IP) experiments. AceCS2 was immunoprecipitated from cell lysates of control and Nampt overexpressing HEK293 cells transfected with control vector or AceCS2-HA for 48 hr.

FIGS. 6A-6F present a schematic equation and graphs to indicate that Nampt regulates total NAD⁺. FIG. 6A presents a schematic showing synthesis of isotope labeled ¹⁸O-NAD⁺, a reference compound used in NAD⁺ measurement. ¹⁸O-NAM was synthesized by hydrolyzing 3-cyanopyridine in ¹⁸O—H₂O, and was then used as a substrate in the enzymatic reaction catalyzed by CD38, a NAD⁺ glycohydrolase, to generate ¹⁸O-NAD⁺. FIGS. 6B-6C present graphs of ion intensity. Total endogenous ¹⁶O-NAD⁺ and spiked-in NAD reference ¹⁸O-NAD⁺ were isolated by HPLC then subjected to MALDI-MS. The ion intensity of the reference peaks of ¹⁸O-NAD⁺ was normalized to 100 in all cases. The ratio of ¹⁶O-NAD⁺ peaks reflects the relative amount of NAD⁺ in the two samples. Experiments were performed at least three times. Total NAD⁺ spectra from HEK293 are shown for vector controls and cells stably-overexpressing Nampt (FIG. 6B) as well as total NAD⁺ spectra from HT1080 vector controls and siRNA-Nampt stable cells (FIG. 6C). FIG. 6D presents a graph of ion intensity indicating that overexpression of Nampt cannot prevent total cellular NAD⁺ depletion by MMS as determined by MALDI-MS spectra of endogenous ¹⁶O-NAD and reference ¹⁸O-NAD after 2 h MMS treatment of HEK293 wildtype and Nampt overexpressing cells. FIG. 6E presents a graph indicating a time course of cell death induced by 1.2 mM MMS treatment. Percent cell death was determined by FACS analysis. FIG. 6F presents a graph indicating total cellular NAD⁺ as measured by MALDI-MS during the time course in (FIG. 6E).

FIGS. 7A-7I present graphs, Western blots and a schematic indicating that mammalian mitochondria maintain mitochondrial NAD⁺ levels during genotoxic stress. FIGS. 7A-7B present ion intensity graphs indicating regulation of mitochondrial NAD⁴ levels by Nampt. Spectra from HEK293 are shown for vector controls and cells stably-overexpressing Nampt (FIG. 7A), as well as spectra from HT1080 vector controls and siRNA-Nampt stable cells (FIG. 7B). FIG. 7C presents an ion intensity graph indicating that additional Nampt greatly attenuates mitochondrial NAD⁺ depletion by MMS treatment, as determined by MALDI-MS after 2 h MMS treatment of HEK293 wildtype and Nampt-overexpressing cells. FIGS. 7D-7E present Western blotting analysis of Nampt in highly purified cytosolic and mitochondrial fractions. Mitochondrial fractions were isolated from HEK293 cells or from rat livers using two different protocols and their purity was assessed by probing for Hsp90, calreticulin, and/or lactate dehydrogenase (exclusively cytoplasmic proteins), and CoxIV or cytochrome C (mitochondrial matrix markers). The same blot was probed for lamin A/C to test for contamination of the mitochondrial fractions with nuclei. The experiment was performed three times on HEK293 cells and on liver tissue. The same pattern was observed each time and representative blots are shown. FIG. 7F presents a schematic showing how mitochondria from rat livers were prepared and exposed to methylmethane sulfonate (MMS), a genotoxic DNA alkylating agent, or the Nampt inhibitor FK866, or both. NAD⁺ levels in isolated mitochondria were determined using MALDI-MS, as above. FIG. 7G presents a graph showing that NAD⁺ levels in isolated mitochondria are reduced by exposure to MMS and FK866. Similar data was obtained using a different mitochondrial isolation protocol. FIG. 7H presents a graph showing that knocking down expression of Nmnat-3 reduces the ability of Nampt to provide resistance to MMS. FIG. 7I presents a graph showing that knocking down expression of a putative human mitochondrial NAD⁺ transporter, hMFT, does not affect survival of Nampt overexpressing cells treated with MMS.

FIGS. 8A-8C present Western blots and a graph indicating that fasting increases hepatic mitochondrial NAD⁺ and Nampt levels. FIG. 8A presents Western blotting analysis indicating that overexpression of Nampt in HEK293 cells inhibits the localization of AIF to the nucleus after MMS treatment for the times indicated. FIG. 8B presents Western blotting analysis of Nampt in mitochondria from rats fed ad libitum or fasted for 48 h. FIG. 8C presents a graph showing relative mitochondrial NAD⁺ levels in liver tissues from rats fed ad libitum or fasted for 48 h. Mitochondrial NAD⁺ levels were measured by MALDI-MS.

FIG. 9 presents a graph showing survival of HEK293 cells after MMS treatment in presence or absence of 30 μM of the PARP-1 inhibitor DPQ. MMS-Induced Cell Death Is Attenuated by Inhibiting PARP-1.

FIG. 10 presents phase-contrast images of HT1080 control cells or Nampt stable knockdown cells treated with MMS for 4 h. Cells with a rounded-up morphology are dying cells, and are more abundant in the cultures of Nampt knockdown cells. Knockdown of Nampt Sensitizes HT1080 Cells to MMS-Induced Cell Death.

FIGS. 11A-11F present Western blots and graphs indicating sirtuin knockdown experiments. FIGS. 11A-11B present Western blots and graphs indicating the effectiveness of sirtuin knockdown by pools of four siRNA oligos (60 nM) targeted against endogenous SIRT1, 2, 3, 5, 7, as assessed by Western Blotting (FIG. 11A) or SIRT4, SIRT6 mRNA (FIG. 11B), as assessed by quantitative RT-PCR. Relative mRNA copy number was determined in comparison to β-actin. FIGS. 11C-11F present graphs indicating survival after treatment with MMS of HEK293 empty vector or Nampt-overexpressing cells transiently transfected with sirtuin siRNA or scrambled siRNA oligos.

FIG. 12 presents a graph and Western blot indicating survival of HEK293 cells after etoposide treatment in cells transfected with siRNA-cont or siRNA-SIRT3 oligos (60 nM). The efficacy of knockdown of SIRT3 by siRNA oligos was assessed by Western blotting.

FIG. 13 presents a graph of mitochondrial NAD⁴. Mitochondria were isolated using the differential centrifugation protocol #2 (see Materials and Methods of Example 2) and incubated for 30 min with methylmethane sulfonate (MMS), FK866, or both. Suspensions were spun-down and analyzed for NAD⁺ content by HPLC-MALDI-MS, using ¹⁸O-NAD⁺ as a reference.

FIG. 14 presents a graph indicating quantitative RT-PCR of Nmnat-3 in siRNA-treated cells. Relative mRNA copy number was determined relative to D-actin.

FIGS. 15A-15B present a sequence alignment and a graph. FIG. 15A shows a sequence alignment of yeast Ndt1 (provided as SEQ ID NO:24) and the putative folate transporter hMFT (provided as SEQ ID NO:25). FIG. 15B shows a graph of quantitative RT-PCR of hMFT in siRNA-treated cells. Relative mRNA copy number was determined in comparison to β-actin.

FIG. 16 presents a phylogenetic comparison of the enzymes that catalyze the first reaction in the salvage of NAD⁺ from nicotinamide (NAM). Unlike S. cerevisiae, C. elegans, and D. melanogaster, which utilize the nicotinamidase Pnc1, vertebrates utilize Nampt, a nicotinamide phosphoribosyltransferase that shares a high degree of homology with enzymes of α-proteobacteria, relatives of the first mitochondria. This may shed light on the evolution of mitochondria during the divergence of the various phyla.

### DETAILED DESCRIPTION

The invention is based at least in part on the discovery that SIRT3 is expressed at high levels in metabolically active tissues and that SIRT3 expression is induced by fasting, caloric restriction and exercise. These results indicate that SIRT3 is involved in regulating energy homeostasis and in responding to physiological and nutritional cues. Modulation of SIRT3 has widespread physiological applications including mimicking the effects of caloric restriction or exercise and increasing metabolism.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The methods described herein may be applied in vitro or in vivo. For example, they may be applied to cells in vitro, either cells from cell lines or cells obtained from a subject.

Increasing SIRT3 may be useful in any subjects in need of metabolic activation of one or more of their muscles, e.g., smooth muscles or cardiac muscles or muscle cells thereof. A subject may be a subject having cachexia or muscle wasting.

Increasing SIRT3 may also be used to increase or maintain body temperature, e.g., in hypothermic subjects. Alternatively, inhibiting SIRT3 may be used to reduce body temperature, e.g., in subjects having fever or hyperthermia.

Increasing SIRT3 may also be used for treating or preventing cardiovascular diseases, reducing blood pressure by vasodilation, increasing cardiovascular health, and increasing the contractile function of vascular tissues, e.g., blood vessels and arteries (e.g., by affecting smooth muscles).

Generally, activation of SIRT3 may be used to stimulate the metabolism of any type of muscle, e.g., muscles of the gut or digestive system, or the urinary tract, and thereby may be used to control gut motility, e.g., constipation, and incontinence. SIRT3 activation may also be useful in erectile dysfunction. It may also be used to stimulate sperm motility, e.g., and be used as a fertility drug.

Other embodiments in which it would be useful to increase SIRT3 include repair of muscle, such as after a surgery or an accident, increase of muscle mass; and increase of athletic performance.

Thus the invention provides methods in which beneficial effects are produced by contacting one or more muscle cells with an agent that increases the protein or activity level of SIRT3 in the cell. These methods effectively facilitate, increase or stimulate one or more of the following: mimic the benefits of calorie restriction or exercise in the muscle cell, increase mitochondrial biogenesis or metabolism, increase mitochondrial activity and/or endurance in the muscle cell, sensitize the muscle cell to glucose uptake, increase fatty acid oxidation in the muscle cell, decrease reactive oxygen species (ROS) in the muscle cell, increase PGC-1α and/or ucp3 and/or GLUT4 expression in the muscle cell, and activate AMP activated protein kinase (AMPK) in the muscle cell.

Various types of muscle cells can be contacted in accordance with the invention. In some embodiments, the muscle cell is a skeletal muscle cell. In certain embodiments, the muscle cell is a cell of a slow-twitch muscle, such as a soleus muscle cell.

The methods of the invention include in some embodiments administering, to a subject in need of such treatment, an agent that increases the protein or activity level of SIRT3 in cells of the subject. A variety of administration methods are known in the art and can be used in the methods described herein. In some embodiments, the agent optionally is targeted to, or administered into, a muscle of the subject.

The agents useful in the aforementioned methods include a SIRT3 protein or biologically effective homolog thereof or a nucleic acid encoding the a SIRT3 protein or biologically effective homolog thereof. In some embodiments, a “biologically effective homolog” is a homolog that has SIRT3 deacetylase and/or SIRT3 ADP ribosyltransferase activity. Examples include fragments of human SIRT3 having deacetylase and/or ADP ribosyltransferase activity.

Other agents useful in the aforementioned methods include small molecules, some of which are described herein, which in some embodiments may also be activators of SIRT1.

The cell that is contacted or the subject that is treated in the aforementioned methods preferably is a cell in need of SIRT3 increase in protein or activity level. In certain embodiments, the cells is a diseased cell of a subject.

Also provided are methods for regulating skeletal muscle metabolism or skeletal muscle energy homeostasis in a subject. In such methods, an agent that modulates the protein or activity level of SIRT3 in the subject, i.e., the SIRT3 modulators described herein, is administered to a subject in need thereof.

Methods for regulating skeletal muscle metabolism or skeletal muscle energy homeostasis in a subject also are provided herein. These methods include administering to a subject in need thereof an agent that modulates the protein or activity level of AMPK, such as an agent that stimulates the protein or activity level of AMPK. For example, stress and exercise induce AMPK activity in skeletal muscle. Insulin-sensitizing drugs of the thiazolidinedione family (activators of PPAR-γ) or metformin can regulate the activity of AMPK. Several hormones secreted by adipocytes (adipokines) either stimulate or inhibit AMPK activation, and may be useful in the aforementioned methods. Leptin and adiponectin are known to stimulate AMPK activation, whereas resistin inhibits AMPK activation.

Also provided are methods for increasing the protein level of SIRT3 in a muscle cell or in muscles of a subject. Such methods include subjecting a cell or a subject to caloric restriction or fasting, or administering to a subject in need thereof an agent that increases the protein or activity level of SIRT3 in a muscle cell. Diseases, disorders and conditions in which such methods are useful include mitochondrial diseases, metabolic disorders, neurologic disorders, muscular disorders, cardiovascular diseases, and excessive weight or obesity. Specific metabolic disorders, diseases or conditions include insulin resistance, diabetes, diabetes related conditions or disorders, or metabolic syndrome. Other metabolic disorders will be known to the skilled person.

Also provided are methods for determining whether a subject has or is likely to develop a disease or condition that is associated with a defect in muscle metabolism or muscle energy homeostasis. In such methods, the level of activity or activity of SIRT3 in a muscle cell of the subject is determined. A statistically significantly lower level of protein or activity of SIRT3 in the cell of the subject relative to a control indicates that the subject has or is likely to develop a disease or condition that is associated with a defect in muscle metabolism or muscle energy homeostasis. As used herein, a control in some embodiments is the level of protein or activity of SIRT3 is a similar muscle cell of a healthy subject, or that of an average of at least 5 healthy subject. In other embodiments, a control can be a range of levels of protein or activity of SIRT3, for example, as based on measurement of a larger number of levels in a larger number of cells.

Also provided are methods for identifying an agent that mimics the benefits of calorie restriction or exercise in a muscle cell. In such methods, a muscle cell is contacted with an agent or the agent is administered to or into a muscle cell (e.g., of a subject in need of such treatment); and it is determined whether the benefits of calorie restriction or exercise are mimicked (as described herein) in the muscle cell by the agent. In some embodiments, the agent increases the protein or activity level of SIRT3 or SIRT3 and SIRT1 in a muscle cell. Thus, in such methods an amount effective to increase the protein or activity level of SIRT3 or SIRT3 and SIRT1 in a muscle cell is used to contact the muscle cell or is administered to or into the muscle cell. The methods in some embodiments also include determining whether the agent increases the protein or activity level of SIRT3 in the muscle cell. In the foregoing methods, the cells in some embodiments are eukaryotic cells, preferably mammalian cells, and more preferably human cells.

SIRT3 activators may also be used to compensate for the effect of certain drugs on muscle or generally, to revitalize a subject after a treatment, e.g., with chemotherapeutic drugs. They may also be used to enhance the performance (e.g., physical performance) of healthy subjects, e.g., subjects that are exposed to certain conditions, e.g., astronauts or subjects in the military.

Cardiovascular diseases that can be treated include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to the retinal arterioles, the glomerular arterioles, the vasa nervorum, cardiac arterioles, and associated capillary beds of the eye, the kidney, the heart, and the central and peripheral nervous systems.

Neurological diseases that can be treated include neurodegencrative diseases. Some non-limiting examples of neurodegenerative disorders include stroke, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, Multiple Sclerosis (MS), and Friedreich's ataxia, Periventricular leukomalacia (PVL), ALS-Parkinson's-Dementia complex of Guam, Wilson's disease, cerebral palsy, progressive supranuclear palsy (Steel-Richardson syndrome), bulbar and pseudobulbar palsy, diabetic retinopathy, multi-infarct dementia, macular degeneration, Pick's disease, diffuse Lewy body disease, prion diseases such as Creutzfeldt-Jakob, Gerstmann-Straussler-Scheinker disease, Kuru and fatal familial insomnia, primary lateral sclerosis, degenerative ataxias, Machado-Joseph disease/spinocerebellar ataxia type 3 and olivopontocerebellar degenerations, spinal and spinobulbar muscular atrophy (Kennedy's disease), familial spastic paraplegia, Wohlfart-Kugelberg-Welander disease, Tay-Sach's disease, multisystem degeneration (Shy-Drager syndrome), Gilles De La Tourette's disease, familial dysautonomia (Riley-Day syndrome), Kugelberg-Welander disease, subacute sclerosing panencephalitis, Werdnig-Hoffmann disease, synucleinopathies (including multiple system atrophy), Sandhoffdisease, conical basal degeneration, spastic paraparesis, primary progressive aphasia, progressive multifocal leukoencephalopathy, striatonigral degeneration, familial spastic disease, chronic epileptic conditions associated with neurodegeneration, Binswanger's disease, and dementia (including all underlying etiologies of dementia). Muscular diseases, including neuromuscular diseases, that can be treated include: muscular dystrophy and myopathy.

Mitochondrial diseases that can be treated include diseases that show a variety of symptoms caused by dysfunction of mitochondria in cells. The mitochondrial disease are classified in various ways by biochemical abnormalities, clinical symptoms or types of DNA abnormalities. Types named as KSS (chronic progressive external ophthalmoplegia), MERRF (myoclonus epilepsy associated with ragged-red fibers; Fukuhara syndrome), MELAS, Leber's disease, Leigh encephalopathia and Pearson's disease are widely known. Among them, MELAS is a type mainly showing stroke-like episodes, occupies 30% or more of the whole and is believed to be the most frequent type in the mitochondrial disease.

Insulin resistance disorders that may be treated include any disease or condition that is caused by or contributed to by insulin resistance. Examples include: diabetes, obesity, metabolic syndrome, insulin-resistance syndromes, syndrome X, insulin resistance, high blood pressure, hypertension, high blood cholesterol, dyslipidemia, hyperlipidemia, dyslipidemia, atherosclerotic disease including stroke, coronary artery disease or myocardial infarction, hyperglycemia, hyperinsulinemia and/or hyperproinsulinemia, impaired glucose tolerance, delayed insulin release, diabetic complications, including coronary heart disease, angina pectoris, congestive heart failure, stroke, cognitive functions in dementia, retinopathy, peripheral neuropathy, nephropathy, glomerulonephritis, glomerulosclerosis, nephrotic syndrome, hypertensive nephrosclerosis some types of cancer (such as endometrial, breast, prostate, and colon), complications of pregnancy, poor female reproductive health (such as menstrual irregularities, infertility, irregular ovulation, polycystic ovarian syndrome (PCOS)), lipodystrophy, cholesterol related disorders, such as gallstones, cholescystitis and cholelithiasis, gout, obstructive sleep apnea and respiratory problems, osteoarthritis, and prevention and treatment of bone loss, e.g. osteoporosis.

Decreasing SIRT3 may be useful in subjects in whom it is beneficial to slow down their metabolism, in particular, the mitochondrial metabolism in muscle cells. These may be subjects having difficulty gaining muscle weight.

As used herein, the terms “increase SIRT3”, “activate SIRT3” and the like mean that the activity of SIRT3 is increased. The activity of SIRT3 can be increased by increasing the activity of the SIRT3 polypeptide and/or by increasing the amount of active SIRT3 polypeptide. Likewise, as used herein, the terms “decrease SIRT3”, “inhibit SIRT3” and the like mean that the activity of SIRT3 is decreased. The activity of SIRT3 can be decreased by decreasing the activity of the SIRT3 polypeptide and/or by decreasing the amount of active SIRT3 polypeptide. Molecules that increase or decrease SIRT3 activity are generically referred to as “SIRT3 modulators”. SIRT3 modulators include SIRT3 activators and SIRT3 inhibitors, any of which may also be referred to herein as “pharmacological agents”, “active compounds”, “components”, “therapeutics” and the like.

In some embodiments the activity or protein level of a sirtuin such as SIRT3 is increased through administering the sirtuin gene or protein. In some embodiments the activity or protein level of a sirtuin such as SIRT3 is increased through administering a compound that increases the protein level or increases the activity a sirtuin. In some embodiments, SIRT3 activators may be any SIRT1 activator that is known in the art that also activates SIRT3. SIRT1 activators are described in numerous U.S. application publications, PCT publications, and references, e.g., as those on which on of the inventors of this application is an inventor or author, all of which are specifically incorporated by reference herein. Methods for activating sirtuins, and non-limiting examples of compounds for activating sirtuins are provided by formulas 1-25, 30, and 32-65 in US Patent Publication 2006/0025337, incorporated by reference herein in its entirety, in particular for these teachings. Methods and compounds for modulating sirtuins are also presented in US Patent Publications: 2007/0043050, 2007/0037865, 2007/0037827, 2007/0037809, 2007/0014833, 2006/0025337, 2006/0276416, 2006/0276393 and 2006/0229265, 2005/0136537, WO 05/002555, WO 2005/065667, WO 2007/084162 and in U.S. Pat. No. 7,345,178, all of which are incorporated herein by reference in their entireties, in particular for these teachings. In other embodiments, SIRT3 activators are specific for SIRT3. Preferably, such SIRT3-specific activators do not activate SIRT1 in a statistically significant amount. Method for measurement of activation of SIRT1 are well known in the art, including in the above-referenced patents and patent publications.

SIRT3 modulators may be administered by any of the known methods, e.g., systemically or locally, topically, intradermally, subcutaneously, intramuscularly, or orally.

GenBank Accession numbers for human SIRT3 and SIRT4 nucleic acids and proteins are as follows:

| | | | |
|---|---|---|---|
| SIRT3 | ia | NM_012239 | NP_036371 |
| | ib | NM_001017524 | NP_001017524 |
| SIRT4 | | NM_012240 | NP_036372 |

### Administration

The pharmacological agents used in the methods of the invention are preferably sterile and contain an effective amount of one or more agents for producing the desired response in a unit of weight or volume suitable for administration to a subject. The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual physician or veterinarian, particularly in the event of any complication. A therapeutically effective amount typically varies from 0.01 mg/kg to about 1000 mg/kg, preferably from about 0.1 mg/kg to about 500 mg/kg, and most preferably from about 0.2 mg/kg to about 250 mg/kg, in one or more dose administrations daily, for one or more days.

Pharmacological agents associated with the invention and optionally other therapeutics may be administered per se or in the form of a pharmaceutically acceptable salt.

Various modes of administration are known to those of ordinary skill in the art which effectively deliver the pharmacological agents of the invention to a desired tissue, cell, or bodily fluid. The administration methods are discussed elsewhere in the application. The invention is not limited by the particular modes of administration disclosed herein. Standard references in the art (e.g, Remington's Pharmaceutical Sciences, 20th Edition, Lippincott, Williams and Wilkins, Baltimore Md., 2001) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration and the like vary from those presented herein.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

The pharmacological agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle (e.g., saline, buffer, or sterile pyrogen-free water) before use.

Compositions suitable for oral administration may be presented as discrete units, such as capsules, tablets, pills, lozenges, each containing a predetermined amount of the active compound. Other compositions include suspensions in aqueous liquids or non-aqueous liquids such as a syrup, elixir, an emulsion, or a gel.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, sorbitol or cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the agent or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, agents may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of an agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery. Agents can be delivered to the lungs of a mammal while inhaling and traverse across the lung epithelial lining to the blood stream. Reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of a given agent. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise an agent dissolved in water at a concentration of about 0.1 to 25 mg of biologically active agent per mL of solution. The formulation may also include a buffer and a simple sugar (e.g., for stabilization and regulation of osmotic pressure). The nebulizer formulation may also contain a surfactant, to reduce or prevent surface induced aggregation of the agent caused by atomization of the solution in forming the aerosol.

Formulations for use with a metered-dose inhaler device will generally comprise a finely divided powder containing the agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing an agent and may also include a bulking agent, such as lactose, sorbitol, sucrose, or mannitol in amounts which facilitate dispersal of the powder from the device, e.g., 50 to 90% by weight of the formulation. The agent should most advantageously be prepared in particulate form with an average particle size of less than 10 mm (or microns), most preferably 0.5 to 5 mm, for most effective delivery to the distal lung.

Nasal (or intranasal) delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic agent(s), may be provided in particles. Particles as used herein means nano or micro particles (or in some instances larger) which can consist in whole or in part of therapeutic agent(s) described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain therapeutic agents described herein in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

For topical administration to the eye, nasal membranes, mucous membranes or to the skin, the therapeutic agents may be formulated as ointments, creams or lotions, or as a transdermal patch or intraocular insert or iontophoresis. For example, ointments and creams can be formulated with an aqueous or oily base alone or together with suitable thickening and/or gelling agents. Lotions can be formulated with an aqueous or oily base and, typically, further include one or more emulsifying agents, stabilizing agents, dispersing agents, suspending agents, thickening agents, or coloring agents. (See, e.g., U.S. Pat. No. 5,563,153, entitled “Sterile Topical Anesthetic Gel”, issued to Mueller, D., et al., for a description of a pharmaceutically acceptable gel-based topical carrier.)

In general, the therapeutic agent is present in a topical formulation in an amount ranging from about 0.01% to about 30.0% by weight, based upon the total weight of the composition. Preferably, the agent is present in an amount ranging from about 0.5 to about 30% by weight and, most preferably, the agent is present in an amount ranging from about 0.5 to about 10% by weight. In one embodiment, the compositions of the invention comprise a gel mixture to maximize contact with the surface of the localized pain and minimize the volume and dosage necessary to alleviate the localized pain. GELFOAM® (a methylcellulose-based gel manufactured by Upjohn Corporation) is a preferred pharmaceutically acceptable topical carrier. Other pharmaceutically acceptable carriers include iontophoresis for transdermal drug delivery.

The invention also contemplates the use of kits. In some aspects of the invention, the kit can include a pharmaceutical preparation vial, a pharmaceutical preparation diluent vial, and one or more therapeutic agents. In some embodiments the kit contains agents for diagnostic purposes such as an antibody or multiple antibodies. The vial containing the diluent for the pharmaceutical preparation is optional. The diluent vial contains a diluent such as physiological saline for diluting what could be a concentrated solution or lyophilized powder of a therapeutic agent. The instructions can include instructions for mixing a particular amount of the diluent with a particular amount of the concentrated pharmaceutical preparation, whereby a final formulation for injection or infusion is prepared. The instructions may include instructions for treating a subject with an effective amount of a therapeutic agent. The instructions may include instructions for diagnosing a patient, characterizing a patient's risk for a given disease, or evaluating the effectiveness of a given therapy for a patient. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized. A kit associated with the invention is presented in FIG. 17.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention.

Accordingly, the foregoing description and drawings are by way of example only.

The present invention is further illustrated by the following examples which should not be construed as limiting in any way. The contents of all cited references (including literature references, issued patents, published patent applications, and GenBank accession numbers as cited throughout this application) are hereby expressly incorporated by reference.

### EXAMPLES

### Example 1

### SIRT3 Regulates Skeletal Muscle Metabolism Through AMPK and PGC-1 Aloha

SIRT3 is a member of the sirtuin family of NAD⁺-dependent deacetylases, which is mainly localized to the mitochondria and is enriched in brown adipose tissue, heart, certain skeletal muscles, and metabolically active tissues. We report here that SIRT3 responds dynamically to nutritional and physiological signals to impact muscle energy homeostasis. We show that murine SIRT3 is more highly expressed in slow oxidative type I soleus muscle compared to the fast type II extensor digitorum longus muscle and gastrocnemius muscle. The muscle protein level of SIRT3 in mice is increased by caloric restriction, fasting, as well as exercise training. Conversely, the SIRT3 expression is repressed in response to three months of high-fat diet feeding. Forced expression of SIRT3 in C2C12 myocytes elevates pgc-1α and ucp3 expression, decreases cellular hydrogen peroxide production, and increases mitochondrial content. Furthermore, the expression of SIRT3 decreases cellular ATP content, increases AMPK activation, and stimulates downstream processes such as glucose uptake and fatty acid oxidation, suggesting that AMPK activation may mediate SIRT3's actions on muscle metabolism.

The sirtuin family of proteins possess NAD⁺-dependent protein deacetylase activity and/or ADP ribosyltransferase activity (1, 2). The seven mammalian sirtuins (SIRT1-SIRT7) are differentially localized within the cell and have diverse functions in regulating cellular action, metabolism, and aging (3, 4). The SIRT3 protein is mainly localized to mitochondria (5-7). Interestingly, SIRT3 has been found to deacetylate and regulate the activity of the mitochondrial enzyme acetyl CoA synthetase 2 (ACS2), which catalyzes the conversion of acetate to acetyl CoA. Glutamate dehydrogenase has also been found to be a substrate of SIRT3, yet the biological significance of this interaction is not known (8). Recent studies suggest that SIRT3 may reside in the nucleus, where it deacetylates histone H4 (9). Furthermore, genetic variations in the human SIRT3 gene have been linked to longevity among certain people (10) and aberrant expression of SIRT3 has been correlated with node-positive breast cancer from clinical biopsies (11), which suggests that SIRT3 may serve as an important diagnostic and therapeutic target in human health and disease.

Indeed, we recently showed that Nampt-mediated cell survival after genotoxic stress promotes maintenance of mitochondrial NAD⁺ levels and requires SIRT3 (and also SIRT4) to block apoptosis (12). Furthermore, in other previous studies, we have shown that the RNA level of SIRT3 is increased by cold exposure and caloric restriction in murine brown adipose tissue and that constitutive expression of SIRT3 in brown pre-adipocytes stimulates the expression of PGC-1α, UCP1, and other mitochondrially-related genes. Functionally, SIRT3 expression in brown adipocytes promotes cellular respiration and reduces both mitochondrial membrane potential and reactive oxygen species (ROS) production (7).

PGC-1α, a nuclear receptor co-activator, plays multiple roles in metabolic regulation (13, 14). It stimulates mitochondrial biogenesis (15), induces muscle fiber-type switch, and increases oxidative capacity in skeletal muscle cells (16). In this study, we have investigated the effects of the SIRT3 gene in skeletal muscle, a key metabolically active organ. Skeletal muscle is the main site for insulin-mediated glucose disposal. Furthermore, skeletal muscle strongly influences whole body lipid metabolism since lipid catabolism provides up to 70% of the energy usage for resting muscle (17). It is also very important to balance fatty acid availability with fatty acid oxidation rates in the muscle, as the intramuscular fatty acid metabolites such as diacylglycerol, causes insulin resistance (18).

AMP activated protein kinase (AMPK) is a key regulator of muscle metabolism. AMPK is a ubiquitous heterotrimeric serine/threonine protein kinase, which functions as a fuel sensor in many tissues, including skeletal muscle (19). AMPK is activated by AMP allosterically and by phosphorylation at Thr172 in the catalytic α-subunit mainly by an upstream AMPK kinase, LKBI (20, 21). Importantly, AMPK is stimulated by cellular stresses that deplete ATP and elevate AMP, such as hypoglycemia (22), exercise (23) and muscular contraction (24). Activated AMPK stimulates ATP-generating catabolic pathways, such as cellular glucose uptake and fatty acid beta-oxidation. AMPK activation also represses ATP-consuming processes, such as lipogenesis to restore intracellular energy balance (19, 25). AMPK not only increases POC-1α expression (26, 27) but also activates PGC-1α by direct phosphorylation (28).

This study shows that SIRT3 levels in skeletal muscle are decreased with high-fat feeding and increased with exercise. Furthermore, forced SIRT3 expression in C2C12 myocytes increases mitochondrial biogenesis, sensitizes the myocytes to glucose uptake, and increases β-oxidation, potentially through the activation of AMPK. Therefore, SIRT3 is likely an important regulator of skeletal muscle metabolism in response to important nutritional and physiological signals.

### Methods

### Animals and Diet

C57BL/6 male mice were used for the studies. For the caloric restriction experiment, C57BL/6 male mice were singly caged. At 8 weeks of age, control mice were fed ad libitum with NIH-31 standard diet (Harlan Teklad, Madison, Wis.), while food consumption was measured daily. Caloric restricted mice were fed with NIH-31/NIA-fortified diet (Harlan Teklad, Madison, Wis.) with a daily food allotment of 90%, 70%, and then 60% of the amount consumed by the control mice—at the first, second, and third week, respectively. From then on, daily food allotment stabilized at 60% of ad libitum food intake for the caloric restricted mice. 12 months later, mice were dissected to collect tissues for analysis. For the fasting experiment, food was removed at 6 μm for 24 hours. For the high-fat diet feeding experiment, 8-week old mice were fed a control or 35% fat diet (Bio-Serv, Frenchtown, N.J.) for three months. Various tissues were also harvested from mice fed the control diet to examine SIRT3 gene expression by Western blot analysis at the termination of the study. For the exercise study (29), 7-week old male and female FVB/NJ mice were wheel cage trained for 6 weeks and fed PicoLab Mouse Diet 20 (LabDiet/Purina, St. Louis, Mo.). In brief, mice were housed in individual cages with or without rodent running wheels (Nalgene, Rochester, N.Y.) and the animals could exercise voluntarily during a 6-week training period. At the end of the 6 weeks, mice were euthanized, triceps muscles was removed, and subsequently analyzed for SIRT3, CREB, phospho-CREB/Ser122, and PGC-1α protein expression (7). Citrate synthase activity was measured as a mitochondrial marker post exercise training from triceps samples, as described previously (29).

### Sirt3 Knock-Out Mice

Mice in which the Sirt3 gene was targeted by gene trapping were obtained from the Texas Institute for Genomic Medicine (Houston, Tex., USA). Briefly, these mice were created by generating embryonic stem (ES) cells (Omnibank no. OST341297) bearing a retroviral promoter trap that functionally inactivates one allele of the Sirt3 gene, as described previously (30). Sequence analysis indicated that retroviral insertion occurred in the intron preceding coding exon 1 (Accession: NM_022433). Targeted 129/SvEvBrd embryonic stem cells were injected into C57BL/6 albino blastocysts. The chimeras (129/SvEvBrd) were then crossed with C57BL/6 albinos to produce the heterozygotes. Heterozygotes were then mated and the offspring were geneotyped using PCR, containing two primers flanking the trapping cassette insertion site TG0003-5′(ATCTCGCAGATAGGCTATCAGC) (SEQ ID NO:1) and TG0003-3′ (AACCACGTAACCTTACCCAAGG) (SEQ ID NO:2), as well as a third primer LTR rev, a reverse primer located at the 5′ end of the trapping cassette (ATAAACCCTCTTGCAGTTGCATC) (SEQ ID NO:3). Primer pair TG0003-5′ and TG0003-3′ amplify a 336 bp fragment from the wildtype allele, while primer pair TG0003-5′ and LTR rev amplify a 160 bp fragment from the knockout allele.

### Cell Culture and Antibodies

C2C12 myocytes and BOSC23 cells were cultured in Dulbecco's modified Eagle's medium containing 10% bovine calf serum. For C2C12 differentiation, at confluence, cells were incubated in Dulbecco's modified Eagle's medium with 2% horse serum (Hyclone, Logan, Utah) for three days. Inhibition of AMPK in selected studies was achieved by adding compound C (6-[4-(2-Piperidin-1-yl-ethoxy)-phenyl)]-3-pyridin-4-yl-pyrrazolo[1,5-a]-pyrimidine, Merck) (31) to the media for 3 hours at a final concentration of 40 μM. The antibodies used for Western blot analysis included: anti-SIRT3 serum antibody against the C-terminus for the tissue distribution and high-fat diet analysis (DLMQRERGKLDGQDR (SEQ ID NO:4), Genemed Synthesis, Inc., San Antonio, Tex.); anti-SIRT3 serum also was developed against the C-terminal region of the protein (Covance, Denver, Pa.), which was validated for specificity using brain, heart, and skeletal muscle tissues from SIRT3 knock-out mice kindly provided by Fred Alt (32), and then used for analyzing the exercise samples; anti-phospho-CREB/Ser133 (Cell Signaling, Danvers, Mass.); anti-CREB (Cell Signaling, Danvers, Mass.); anti-GLUT4 (kindly donated by Paul F. Pilch, Boston University School of Medicine); anti-phospho-AMPK (Cell Signaling, Danvers, Mass.); AMPK (Cell Signaling, Danvers, Mass.); anti-phospho-acetyl CoA carboxylase (UpState Inc. Charlottesville, Va.); anti-PGC-1α (Calbiochem, San Diego, Calif.); Cytochrome C Oxidase subunit IV (COX IV) (Invitrogen, Carlsbad, Calif.); 3-actin antibody (Santa Cruz Biotechnology, Santa Cruz, Calif.); and α-tubulin (Abcam, Cambridge, Mass.).

### Retroviral Infection

For retroviral production with pBabe-puro, constructs containing vector alone (Vector), wild-type murine SIRT3 (SIRT3), murine SIRT3 with a G11A mutation in its ADP-ribosyltransferase activity or murine SIRT3 with a N87A mutation in its deacetylase activity were previously designed and characterized by our laboratory (7). The viral particles were used to infect C2C12 myocytes. The cells were then selected by 2 μg/ml puromycin.

### Northern Blot Analysis

The experiments were performed as previously described (7).

Real-Time Quantitative PCR Analysis of Mitochondrial DNA (mtDNA) Content

Total DNA was isolated from C2C12 cells using the salting-out method (33). Ten nanograms of total DNA was used in Q-PCR assay, similarly to the published procedures (34). The ND1 region of mouse mtDNA (GI:34538597) was amplified using forward primer 5′ TCTAATCGCCATAGCCTTCC3′ (SEQ ID NO:5) and reverse primer 5′GGTTGTTAAAGGGCGTATTGG′ (SEQ ID NO:6), giving a fragment of 158 bp; a fragment of mouse beta 2 microglobulin (B2M) gene (GI:149338249) was amplified using forward primer 5′ TGATGGTGAGGTCTGGAATG3′ (SEQ ID NO:7) and reverse primer 5′ GCAGGTTCAAATGAATCTTCAG3′ (SEQ ID NO:8) giving a fragment of 108 bp. The mtDNA content (mtDNA/B2M ratio) was calculated using the formula: mtDNA content=½^{ΔCt}, where ΔCt=Ct^{mDNA}−Ct^{B2M}.

Glucose Uptake

C2C12 cells containing Vector or SIRT3 were differentiated with 2% horse serum for three days, followed by an overnight incubation in DMEM containing 0.5% albumin. The following day, cells were incubated with Krebs Ringer Hepes buffer in the absence or presence of 100 nM insulin for 20 min. The glucose transport assay was initiated by the addition of 100 μM 2-deoxy-D-glucose (plus 0.5 μCi 2-deoxy-D-[³H]glucose). Glucose uptake was terminated by washing the cells in ice-cold Krebs Ringer Hepes buffer. Cells were lysed with lysis buffer (0.1% SDS and 200 mM NaOH) and aliquots were taken to determine radioactivity and protein content. Glucose uptake was normalized to total protein content.

### Glucose, Leucine, and Palmitate Oxidation

C2C12 cells containing Vector or SIRT3 were differentiated with 2% horse serum for three days in 25-cm² flasks. On the final day, myocytes were cultured in the presence of glucose (25 mM), leucine (1 mM), and palmitate (0.4 mM); the radiolabel tracers (200 nCi/ml) [1-¹⁴C]glucose, [1-¹⁴C]leucine, or [1-¹⁴C]palmitate (Amersham, Piscataway, N.J.) were added in separate experiments for determination of glucose, leucine, and palmitate oxidation, respectively. The flasks were sealed and fitted with a center well containing a piece of Whatman filter paper, glued to a cap. After 6 h, the ¹⁴CO_{z} was released from the culture medium by acidification with 2 ml of 6 N HCl, and the filter paper saturated with 250 μl of 2 N NaOH. ¹⁴CO₂ absorbed on the paper filter was measured by scintillation counting.

### Measurement of Hydrogen Peroxide Production

C2C12 cells containing Vector or SIRT3 were differentiated with 2% horse serum for 3 days. Cells were harvested in PBS and subjected to 3 freeze-thaw cycles to lyse cells. Total hydrogen peroxide (H₂O₂) levels were detected using the Amplex Red Hydrogen Peroxide/Peroxidase Assay Kit following the manufacturer's protocol (Invitrogen, Carlsbad, Calif.).

### Determination of Mitochondrial Membrane Potential (ΔΨm)

The fluorescent probe 5,5′,6,6′-tetrachloro-1,1′,3,3′-tetraethylbenzimidazole carbocyanide iodide (JC-1; Invitrogen) was used to measure cellular ΔΨm. Briefly, C2C12 cells (1×10⁶) were harvested by trypsinization. After washing with PBS, cells were incubated with 10 μg/ml JC-1 at 37° C. for 15 min. Cells were then washed in PBS, 100 μl cell suspension was transferred into wells of black 96-well plate. The red fluorescence (excitation 550 nm, emission 590 nm) and green fluorescence (excitation 485 nm, emission 530 nm) were measured using a fluoremeter (SpectraMAX GEMINIXS, Molecular Devices). Data were presented as a ratio of relative red/green (aggregate/monomer) fluorescence intensity values.

### Measurement of Cellular ATP

Differentiated C2C12 cells expressing either Vector or SIRT3 were rinsed once with PBS and lysed with a 2.5% (w/v) trichloroacetic acid (TCA) solution for 5 minutes. A 10-fold volume of 250 mM Tris base (pH 7.75 using acetic acid) was added to the TCA cell slurry solution to neutralize the acid. The Enliten ATP assay system bioluminescence detection kit (Promega, Madison, Wis.) was used following manufacturer's protocol to detect total cellular ATP levels.

### Measurement of Glycogen Synthesis and Lactate Release

C2C12 cells were differentiated and the cells were treated with ¹⁴C glucose (1 μCi/ml) in the presence of 5 μg/ml of insulin for 6 hours. To determine the glycogen production, cell pellets were digested in 1M KOH. Saturated Na₂SO₄, ethanol and glycogen were added. After centrifugation at 20,000 g for 10 minutes, the pellet was washed with 70% ethanol and dissolved in water and counted on a scintillation counter. Lactate released into medium was measured by ion exchange columns. Briefly, collected cell culture media were added to a column prepared from 200-400 mesh AG1-X8 resin (Bio-Rad, Hercules, Calif.). After multiple washes with 5 mM glucose, ¹⁴C lactate was eluted using a 1 μM lactate and 0.5M formic acid solution and counted on a scintillation counter.

### Results

We first examined the protein expression pattern of SIRT3 in various mouse tissues using a rabbit host polyclonal antibody against the last 15 amino acid C-terminal residues of murine SIRT3. The SIRT3 protein tissue distribution pattern mirrors that of SIRT3 mRNA (7). SIRT3 exhibits an interesting distribution pattern with high expression in key metabolically active tissues, such as brown fat, liver, brain, certain muscle and kidney. Interestingly, SIRT3 protein levels were higher in the slow-twitch soleus muscle compared to the fast twitch muscles (extensor digitorum longus and gastrocnemius). Since the soleus muscle is composed predominantly of type I fiber types relying on fatty acid oxidative metabolism (35), SIRT3 may have an important role in oxidative metabolism in muscle. We had previously shown that caloric restriction stimulates the expression of SIRT3 in brown fat (7). In this study, we found the SIRT3 protein amount was increased in the gastrocnemius and quadriceps when the mice were calorie restricted at 60% of ad libitum for twelve months. In addition, twenty-four hours of fasting also induced the expression of SIRT3 in the extensor digitorum longus muscle. Conversely, following three months of high-fat feeding, the protein level of SIRT3 in the hind limb muscle was significantly decreased.

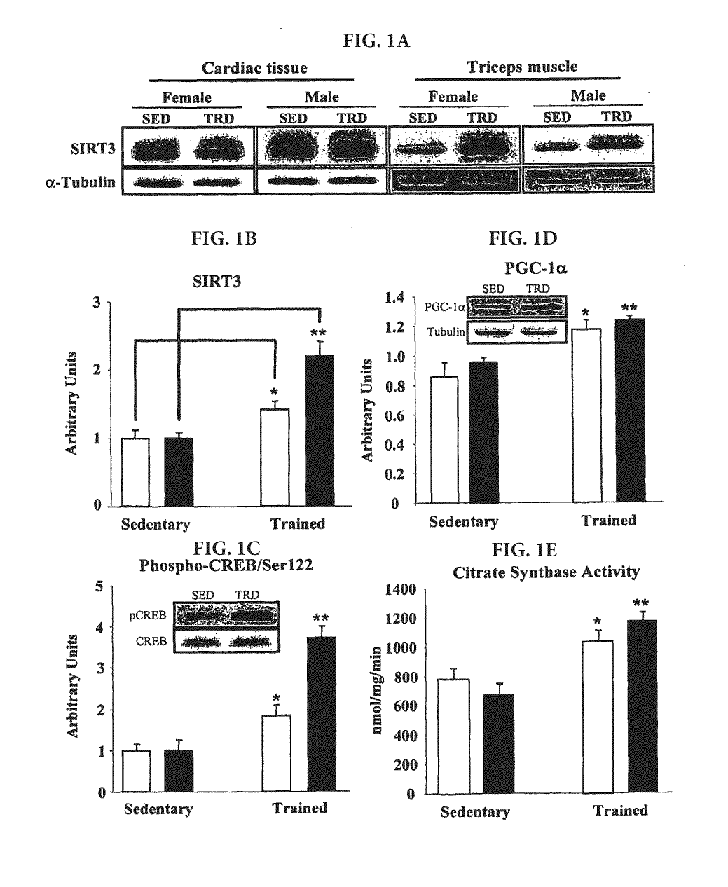

Additionally, we found that SIRT3 protein expression was increased selectively in triceps of exercise trained mice after a six-week training period, with no significant change observed in cardiac muscle samples tested from those same animals (FIG. 1A). Although we did find SIRT3 enriched in heart tissue, it was not altered by exercise. Strikingly, induction of SIRT3 in skeletal muscle was more dramatic in female mice when compared to that of male littermates (FIG. 1B). Furthermore, this increased SIRT3 expression correlated strongly with enhanced CREB-specific phosphorylation at Ser133 and PGC-1α induction in triceps (FIGS. 1C and 1D, respectively)—phenomena that we previously observed in HIBIB brown adipocytes due to elevated SIRT3(7). As a mitochondrial marker following the exercise training, citrate synthase activity was measured (FIG. 1E). Interestingly, skeletal muscle-specific induction of SIRT3 was also observed as early as 2 weeks into training with rats on a previously described treadmill-based exercise paradigm (36) (data not shown). Lastly, upon closer analysis at lighter exposures, the protein banding pattern of SIRT3 in heart lysates differed significantly from that in triceps (FIG. 1A), suggesting that there may exist alternative post-translational processing of SIRT3 in these two tissues, which may contribute to different biological roles for SIRT3 within tissue- and/or metabolic-specific contexts.

Together these data suggested a possible link between SIRT3 expression and alterations in muscle metabolism, specifically in response to caloric intake and exercise. Thus, to further determine and test the downstream effects of SIRT3 levels on muscle metabolism, we used a retroviral expression system to stably express SIRT3 in the murine muscle cell line C2C12 in vitro. We infected cells with either a vector control, wild-type SIRT3, or mutants in which the ADP-ribosyltransferase activity was inactivated (G11A) or in which the NAD⁺-dependent deacetylase activity was removed (N87A), as described previously (7). Since we had already shown that SIRT3 is able to increase the expression of PGC-1α and UCP1 in brown adipocytes, we first determined whether SIRT3 may have a similar effect in C2C12 myocytes. We found that increased expression of SIRT3 in C2C12 cells elevated levels of both pgc-1α and ucp3. It is important to note that increased UCP3 expression in muscle protects against ROS damage and may function to facilitate fatty acid oxidation (37). Strikingly, pre-diabetic and diabetic individuals have decreased skeletal muscle expression of UCP3 (38). In agreement with the elevation of ucp3 expression, we observed decreased mitochondrial membrane potential and decreased cellular hydrogen peroxide level in SIRT3-expressing C2C12 cells. Increased PGC-1α has been shown to result in increased mitochondrial biogenesis in skeletal muscle (15). Therefore, we measured mitochondrial DNA content using quantitative PCR analysis and found that SIRT3 caused increased mitochondrial DNA content in differentiated C2C12 cells. In addition, the expression of mitochondrial proteins, such as cytochrome C oxidase subunit IV (COX IV), was also increased in the SIRT3 over-expressing C2C12 cells.

Since SIRT3 increased PGC-1α expression in C2C12 myocytes and PGC-1α expression is associated with increased muscle glucose uptake (39), we hypothesized that glucose uptake would also be increased by SIRT3. Our results indicate that constitutive expression of SIRT3 elevates both basal and insulin-stimulated glucose uptake in C2C12 myocytes. This observation is also reflected by an elevation of GLUT4 protein level in SIRT3 over expression cells. Interestingly, glucose oxidation was not enhanced due to increased glucose uptake, as glucose oxidation rates did not change in cells expressing SIRT3. In addition, SIRT3 over-expression had no effect on leucine oxidation in C2C12 myotubes, an indicator of amino acid catabolism. However, fatty acid oxidation was drastically increased in C2C12 myotubes expressing SIRT3, as compared to the vector control, further supporting a unique role for SIRT3 in oxidative lipid metabolism. Since SIRT3 increases glucose uptake without increasing glucose oxidation, we reasoned that enhanced glycogen deposition and lactate release could be likely destinations for the glucose. To test this hypothesis, we measured ¹⁴C incorporation into glycogen and lactate after six hour incubation of cells with [¹⁴C]-glucose. SIRT3 significantly increases glycogen synthesis and lactate release.

The induction of PGC1-1α and GLUT4 expression, and increased rates of glucose uptake and fatty acid oxidation, are all phenomenon commonly observed with increased AMPK activation (26, 27). Therefore we measured the effect of SIRT1 expression on AMPK phosphorylation at the Thr172 site of the alpha subunit, an indicator of AMPK activity. Results confirmed that SIRT3 is a positive regulator of AMPK activity in C2C12 cells. Since AMPK is activated by an increase of the AMP/ATP ratio, and given that SIRT3 resides in mitochondria—a key site of ATP production, we next measured the cellular levels of ATP. We discovered that increased expression of SIRT3 decreased total cellular ATP levels in C2C12 cells, thereby providing a potential mechanism by which SIRT3 activates AMPK (and associated downstream processes). To confirm that AMPK activation may mediate the effects of SIRT3 on glucose and lipid metabolism, we treated C2C12 myotubes with 40 μM compound C, an AMPK inhibitor (31). A 3-hour treatment of compound C decreased AMPK phosphorylation, in both vector- and SIRT3-transfected cells. Strikingly, the ability of SIRT3 to increase GLUT4 protein expression was completely abolished by compound C, thereby clearly demonstrating that the increase in GLUT4 expression by SIRT3 requires AMPK activity.

Finally, to confirm the link between SIRT3 and AMPK in vivo, we examined AMPK phosphorylation in the quadriceps of caloric restricted mice, where we showed that SIRT3 protein levels increase. Consistent with the C2C12 data, the AMPK phosphorylation level was increased accordingly in the muscles of caloric restricted mice. Most importantly, we investigated the state of AMPK activation in the skeletal muscles of SIRT3 knock-out mice. We obtained SIRT3 knock-out mice from the Texas Institute for Genomic Medicine, in which the SIRT3 deficiency was confirmed by Western blot analysis, using multiple tissue samples. AMPK phosphorylation was significantly decreased in the EDL muscle of the SIRT3 knock-out mice. In addition, the expression of PGC-1α mRNA was decreased in the gastrocnemius muscle of SIRT3 heterozygous or homozygous deficient mice. Collectively, these data provide strong evidence that SIRT3 is induced by a reduction in caloric intake or exercise training, and that the induction of SIRT3 promotes glucose uptake and 1-oxidation via activation of AMPK.

### Discussion

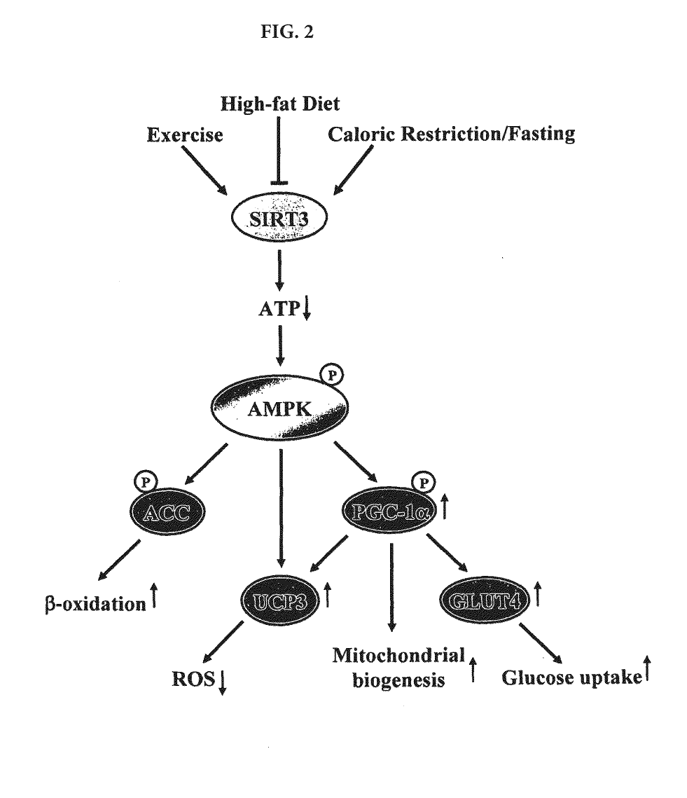

In summary, we have found that SIRT3 is differentially expressed in mouse tissues with greatest expression observed in metabolically active tissues such as soleus muscle. SIRT3 expression in muscle is decreased by high-fat feeding, while increased by short-term (24-hour fasting) or long-term nutrient deprivation (12-month caloric restriction) and exercise training. Increased SIRT3 expression in C2C12 cells is shown to decrease cellular ATP levels and activate AMPK, which in turn mediates metabolic and gene expression effects observed—including induced PGC-1α and UCP3 expression, increased rates of glucose uptake, and fatty acid oxidation (FIG. 2). Thus, our findings show that skeletal muscle SIRT3 levels change in response to physiological and nutritional alterations to regulate muscle energy homeostasis. Our data are also consistent with SIRT3 mediating the physiological changes and health benefits of diet restriction and exercise.

The increase in SIRT3 expression that we observed during nutrient deprivation and exercise may facilitate glucose uptake and fatty acid metabolism to maintain the adequate function of muscle, while the decrease in SIRT3 protein during high-fat feeding may contribute to intramuscular lipid accumulation and insulin resistance. The down-regulation of SIRT3 expression could be a direct response to high-fat feeding or a secondary response to obesity and associated metabolic abnormalities induced by three months of excessive energy intake. A recent study of SIRT3-deficient mice did not find any defect in basal metabolism or adaptive thermogenesis under standard diet and sedentary housing conditions (8). Given our findings, it will be interesting to test whether these mice show metabolic defects on a high-fat diet or are less responsive to exercise. There may also be functional redundancy among sirtuins, such that the effect of loss of SIRT3 is less apparent in the knock-out animal. In this case, it may be interesting to generate a mouse model with increased expression of SIRT3 in muscle to further test our hypotheses.

The exact mechanism by which mitochondrial SIRT3 decreases cellular ATP levels and activates AMPK in the myocyte is not clear. Based on current knowledge, one way that this may occur is through SIRT3-mediated deacetylation and subsequent activation of acetyl CoA synthetase 2 (ACS2) (40, 41). The utilization of acetate by ACS2 is an energy-consuming process. Acetyl CoA synthetase 2 utilizes two high energy phosphates from ATP to metabolize acetate to acetyl CoA resulting in AMP formation. Therefore, increased SIRT3 expression is likely to increase the activity of acetyl CoA synthetase 2 may subsequently elevate the AMP/ATP ratio. SIRT3 may also act through additional, unidentified targets, resulting in reduced ATP levels. It is interesting that SIRT3 increases mitochondrial biogenesis and fatty acid oxidation but also decreases ATP levels. This may be explained by the fact that SIRT3 promotes UCP3 expression and decreases mitochondrial membrane potential, which may result in uncoupling of oxidative phosphorylation and decrease of ATP production. Although it is controversial regarding the action of UCP3, there are evidences that either over expression or knockout of UCP3 resulted in the changes of mitochondrial coupling or ATP synthesis in skeletal muscle (42, 43). Furthermore, in light that SIRT3 deficiency elevates the acetylation levels of multiple mitochondrial proteins (8), SIRT3 may activate other ATP consuming processes or suppress ATP producing processes.

We have discovered that AMPK is activated when SIRT3 level is elevated by retroviral expression. This observation suggested that AMPK might mediate the metabolic effects of SIRT3. It is known that AMPK directly phosphorylates POC-1α (28) and CREB (44), both of which are involved in the transactivation of PGC-1α (45, 46). In addition, AMPK also activates the expression of GLUT4 through PGC-1α (28). Our findings that SIRT3 activates AMPK and glycogen synthesis seem contradictory to reports that AMPK phosphorylates and inactivates glycogen synthase (47, 48), although there are also reports showing that activation of AMPK by AICAR resulted in the increase of glycogen content (49, 50). In addition, AMPK mutants with increased AMPK activity also caused an increase of glycogen content in the skeletal muscle of transgenic mice (51, 52) or human patients (53). In one study with transgenic expression of one of such AMPK activating mutant in the heart resulted in the increase of glucose uptake, fatty acid oxidation, and glycogen synthesis (54), quite similar to our results. Therefore, although AMPK may phosphorylate and inhibit glycogen synthase, in vivo the effect could be overridden by the increase of glucose uptake and accompanying increase of glucose-6-phosphate, which activates glycogen synthase allosterically (55). In addition, glycogen synthase activity is also regulated by other kinases and phosphatases.

AMPK activation may result in lifespan extension (56-58) and therefore may be a potential mechanism by which caloric restriction-mediated activation of sirtuins may increase longevity. The ability to activate AMPK may not be specific to SIRT3 since SIRT1 is also able to activate AMPK (59). Furthermore, resveratrol, a known SIRT1 activator, also enhances AMPK activation and PGC-1α expression (60). It is also noteworthy that SIRT3 shares a similar metabolic regulatory function in skeletal muscle as nuclear SIRT1 (61). Both SIRT3 and SIRT1 promote mitochondrial biogenesis and fatty acid oxidation. However, SIRT3 and SIRT1 activate PGC-1α function in different manner. SIRT3 increases PGC-1α expression, while SIRT1 activates POC-1α by deacetylation (61); therefore, the two proteins may work cooperatively within certain biological contexts.

The ability of a mitochondrial sirtuin to activate AMPK and downstream targets such as PGC-1α, glucose uptake, and β-oxidation may have several therapeutic implications, including metabolic syndrome, obesity, and Type 2 diabetes. High oxidative capacity in muscle is important in maintaining muscle fuel source flexibility, and has been positively correlated with insulin sensitivity (16, 62, 63). Increasing evidence supports the hypothesis that mitochondrial dysfunction may be a key mediator between cellular oxidative metabolic dysfunction and insulin resistance, leading to Type 2 diabetes (64). Recent studies have linked the reduction of muscle mitochondrial oxidative phosphorylation activity to insulin resistance in young and aged patients (65, 66). Increases in mitochondrial ROS production have been linked to high-fat diets, insulin resistance, and obesity (67). Furthermore, insulin-resistant subjects were found to have a lower ratio of oxidative type I muscle fibers as compared to controls, indicating decreased oxidative metabolism. They also have decreased expression of PGC-1α, PGC-1β, and related genes involved in oxidative phosphorylation (68, 69). Since SIRT3 is able to increase the expression of genes involved in oxidative metabolism and decrease reactive oxygen species formation, increase lipid oxidative metabolism, and increase both insulin-dependent and insulin-independent glucose uptake, activators of SIRT3 may be an important therapeutic route for the future treatment of metabolic disorders, obesity, and Type 2 diabetes. Lastly, the recent finding that PGC-1α is important for preventing neurodegenerative diseases (70, 71) raises the possibility that SIRT3 activation may also be of therapeutic value within neurodegenerative-type disorders.

### ABBREVIATIONS

The abbreviations used are: ACS 2, acetyl CoA synthetase 2; AMPK, AMP activated protein kinase; COX IV, cytochrome C oxidase subunit IV; GLUT4, glucose transporter 4; PGC-1α, PPARγ coactivator 1α; ROS, reactive oxygen species; SIRT3, sirtuin 3; UCP1, uncoupling protein 1; UCP3, uncoupling protein 3.

### REFERENCES FOR EXAMPLE 1

1. Haigis, M. C., and Guarente, L. P. (2006) Mammalian sirtuins—emerging roles in physiology, aging, and calorie restriction. Genes Dev 20, 2913-2921
2. Michan, S., and Sinclair, D. (2007) Sirtuins in mammals: insights into their biological function. Biochem J 404, 1-13
3. Michishita, E., Park, J. Y., Burneskis, J. M., Barrett, J. C., and Horikawa, I. (2005) Evolutionarily conserved and nonconserved cellular localizations and functions of human SIRT proteins. Mol Blol Cell 16, 4623-4635
4. North, B. J., and Verdin, E. (2004) Sirtuins: Sir2-related NAD-dependent protein deacetylases. Genome Biol 5, 224
5. Onyango, P., Celic, I., McCaffery, J. M., Boeke, J. D., and Feinberg, A. P. (2002) SIRT3, a human SIR2 homologue, is an NAD-dependent deacetylase localized to mitochondria. Proc Natl Acad Sci USA 99, 13653-13658
6. Schwer, B., North, B. J., Frye, R. A., Ott, M., and Verdin, E. (2002) The human silent information regulator (Sir)2 homologue hSIRT3 is a mitochondrial nicotinamide adenine dinucleotide-dependent deacetylase. J Cell Biol 158, 647-657
7. Shi, T., Wang, F., Stieren, E., and Tong, Q. (2005) SIRT3, a mitochondrial sirtuin deacetylase, regulates mitochondrial function and thermogenesis in brown adipocytes. J Biol Chem 280, 13560-13567
8. Lombard, D. B., Alt, F. W., Cheng, H.-L., Bunkenborg, J., Streeper, R. S., Mostoslavsky, R., Kim, J., Yancopoulos, G., Valenzuela, D., Murphy, A., Yang, Y., Chen, Y., Hirschey, M. D., Bronson, R. T., Haigis, M., Guarente, L. P., Farese, R. V., Jr., Weissman, S., Verdin, E., and Schwer, B. (2007) Mammalian Sir2 Homolog SIRT3 Regulates Global Mitochondrial Lysine Acetylation 10.1128/MCB.01636-07. Mol. Cell. Blol. 27, 8807-8814
9. Scher, M. B., Vaquero, A., and Reinberg, D. (2007) SirT3 is a nuclear NAD⁺ -dependent histone deacetylase that translocates to the mitochondria upon cellular stress 10.1101/gad.1527307. Genes Dev. 21, 920-928
10. Rose, G., Dato, S., Altomare, K., Bellizzi, D., Garasto, S., Greco, V., Passarino, G., Feraco, E., Mari, V., Barbi, C., BonaFe, M., Franceschi, C., Tan, Q., Boiko, S., Yashin, A. I., and De Benedictis, G. (2003) Variability of the SIRT3 gene, human silent information regulator Sir2 homologue, and survivorship in the elderly. Exp Gerontol 38, 1065-1070
11. Ashraf, N., Zino, S., Macintyre, A., Kingsmore, D., Payne, A. P., George, W. D., and Shiels, P. G. (2006) Altered sirtuin expression is associated with node-positive breast cancer. Br J Cancer 95, 1056-1061
12. Yang, H., Yang, T., Baur, J. A., Perez, E., Matsui, T., Carmona, J. J., Lamming, D. W., Souza-Pinto, N. C., Bohr, V. A., Rosenzweig, A., de Cabo, R., Sauve, A. A., and Sinclair, D. A. (2007) Nutrient-Sensitive Mitochondrial NAD(+) Levels Dictate Cell Survival. Cell 130, 1095-1107
13. Finck, B. N., and Kelly, D. P. (2006) PGC-1 coactivators: inducible regulators of energy metabolism in health and disease 10.1172/JCI27794. J. Clin. Invest. 116, 615-622
14. Handschin, C., and Spiegelman, B. M. (2006) Peroxisome Proliferator-Activated Receptor {gamma} Coactivator 1 Coactivators, Energy Homeostasis, and Metabolism 10.1210/er.2006-0037. Endocr Rev, er.2006-0037
15. Wu, Z., Puigserver, P., Andersson, U., Zhang, C., Adelmant, G., Mootha, V., Troy, A., Cinti, S., Lowell, B., Scarpulla, R. C., and Spiegelman, B. M. (1999) Mechanisms controlling mitochondrial biogenesis and respiration through the thermogenic coactivator PGC-1. Cell 98, 115-124
16. Lin, J., Wu, H., Tanr, P. T., Zhang, C. Y., Wu, Z., Boss, O., Michael, L. F., Puigserver, P., Isotani, E., Olson, E. N., Lowell, B. B., Bassel-Duby, R., and Spiegelman, B. M. (2002) Transcriptional co-activator PGC-1 alpha drives the formation of slow-twitch muscle fibres. Nature 418, 797-801
17. Smith, A. G., and Muscat, G. E. (2005) Skeletal muscle and nuclear hormone receptors: implications for cardiovascular and metabolic disease. Int J Biochem Cell Biol 37, 2047-2063
18. Yu, C., Chen, Y., Cline, G. W., Zhang, D., Zong, H., Wang, Y., Bergeron, R., Kim, J. K., Cushman, S. W., Cooney, G. J., Atcheson, B., White, M. F., Kraegen, E. W., and Shulman, G. I. (2002) Mechanism by Which Fatty Acids Inhibit Insulin Activation of Insulin Receptor Substrate-1 (IRS-1)-associated Phosphatidylinositol 3-Kinase Activity in Muscle 10.1074/jbc.M200958200. J. Biol. Chem. 277, 50230-50236
19. Kahn, B. B., Alquier, T., Carling, D., and Hardie, D. G. (2005) AMP-activated protein kinase: ancient energy gauge provides clues to modern understanding of metabolism. Cell Metab 1, 15-25
20. Hawley, S. A., Boudeau, J., Reid, J. L., Mustard, K. J., Udd, L., Makela, T. P., Alessi, D. R., and Hardie, D. G. (2003) Complexes between the LKBI tumor suppressor, STRADalpha/beta and MO25alpha/beta are upstream kinases in the AMP-activated protein kinase cascade. J Biol 2, 28
21. Woods, A., Johnstone, S. R., Dickerson, K., Leiper, F. C., Fryer, L. G., Neumann, D., Schlattncr, U., Wallimann, T., Carlson, M., and Carling, D. (2003) LKBI is the upstream kinase in the AMP-activated protein kinase cascade. Curr Biol 13, 2004-2008
22. Itani, S. I., Saha, A. K., Kurowski, T. G., Coffin, H. R., Tornheim, K., and Ruderman, N. B. (2003) Glucose Autoregulates Its Uptake in Skeletal Muscle: Involvement of AMP-Activated Protein Kinase. Diabetes 52, 1635-1640
23. Fujii, N., Hayashi, T., Hirshman, M. F., Smith, J. T., Habinowski, S. A., Kaijser, L., Mu, J., Ljungqvist, O., Birnbaum, M. J., Witters, L. A., Thorell, A., and Goodyear, L. J. (2000) Exercise induces isoform-specific increase in 5′AMP-activated protein kinase activity in human skeletal muscle. Biochem Biophys Res Commun 273, 1150-1155
24. Hutber, C. A., Hardie, D. G., and Winder, W. W. (1997) Electrical stimulation inactivates muscle acetyl-CoA carboxylase and increases AMP-activated protein kinase. Am J Physiol 272, E262-266
25. Kemp, B. E., Stapleton, D., Campbell, D. J., Chen, Z. P., Murthy, S., Walter, M., Gupta, A., Adams, J. J., Katsis, F., van Denderen, B., Jennings, I. G., Iseli, T., Michell, B. J., and Witters, L. A. (2003) AMP-activated protein kinase, super metabolic regulator. Biochem Soc Trans 31, 162-168
26. Bergeron, R., Ren, J. M., Cadman, K. S., Moore, I. K., Perret, P., Pypaert, M., Young, L. H., Semenkovich, C. F., and Shulman, G. I. (2001) Chronic activation of AMP kinase results in NRF-1 activation and mitochondrial biogenesis. Am J Physiol Endocrinol Metab 281, E1340-1346
27. Zong, H., Ren, J. M., Young, L. H., Pypaert, M., Mu, J., Birnbaum, M. J., and Shulman, G. I. (2002) AMP kinase is required for mitochondrial biogenesis in skeletal muscle in response to chronic energy deprivation. Proc Natl Acad Sci US A 99, 15983-15987
28. Jager, S., Handschin, C., St-Pierre, J., and Spiegelman, B. M. (2007) AMP-activated protein kinase (AMPK) action in skeletal muscle via direct phosphorylation of PGC-1{alpha}. Proc Natl Acad Sci USA 104, 12017-12022
29. Rockl, K. S., Hirshman, M. F., Brandauer, J., Fujii, N., Witters, L. A., and Goodyear, L. J. (2007) Skeletal muscle adaptation to exercise training: AMP-activated protein kinase mediates muscle fiber type shift. Diabetes 56, 2062-2069
30. Zambrowicz, B. P., Friedrich, G. A., Buxton, E. C., Lilleberg, S. L., Person, C., and Sands, A. T. (1998) Disruption and sequence identification of 2,000 genes in mouse embryonic stem cells. 392, 608-611
31. Zhou, G., Myers, R., Li, Y., Chen, Y., Shen, X., Fenyk-Melody, J., Wu, M., Ventre, J., Doebber, T., Fujii, N., Musi, N., Hirshman, M. F., Goodyear, L. J., and Moller, D. E. (2001) Role of AMP-activated protein kinase in mechanism of metformin action. J Clin Invest 108, 1167-1174
32. Lombard, D. B., Alt, F. W., Cheng, H. L., Bunkenborg, J., Streeper, R. S., Mostoslavsky, R., Kim, J., Yancopoulos, G., Valenzuela, D., Murphy, A., Yang, Y., Chen, Y., Hirschey, M. D., Bronson, R. T., Haigis, M., Guarente, L. P., Farese, R. V., Jr., Weissman, S., Verdin, E., and Schwer, B. (2007) Mammalian Sir2 Homolog SIRT3 Regulates Global Mitochondrial Lysine Acetylation. Mol Cell Biol
33. Lahiri, D. K., and Nurnberger, J. I., Jr. (1991) A rapid non-enzymatic method for the preparation of HMW DNA from blood for RFLP studies. Nucleic Acids Res 19, 5444
34. Bai, R. K., Perng, C. L., Hsu, C. H., and Wong, L. J. (2004) Quantitative PCR analysis of mitochondrial DNA content in patients with mitochondrial disease. Ann N YAcad Sci 1011, 304-309
35. Storlien, L., Oakes, N. D., and Kelley, D. E. (2004) Metabolic flexibility. Proc Nutr Soc 63, 363-368
36. Koh, H. J., Hirshman, M. F., He, H., Li, Y., Manabe, Y., Balschi, J. A., and Goodyear, L. J. (2007) Adrenaline is a critical mediator of acute exercise-induced AMP-activated protein kinase activation in adipocytes. Biochem J 403, 473-481
37. MacLellan, J. D., Gerrits, M. F., Gowing, A., Smith, P. J., Wheeler, M. B., and Harper, M. E. (2005) Physiological increases in uncoupling protein 3 augment fatty acid oxidation and decrease reactive oxygen species production without uncoupling respiration in muscle cells. Diabetes 54, 2343-2350
38. Schrauwen, P., Mensink, M., Schaart, G., Moonen-Kornips, E., Sels, J. P., Blaak, E. E., Russell, A. P., and Hesselink, M. K. (2005) Reduced skeletal muscle UCP3 protein content in pre-diabetic subjects and type 2 diabetic patients: restoration by rosiglitazone treatment. J Clin Endocrinol Metab
39. Oliveira, R. L. G. S., Ueno, M., de Souza, C. T., Pereira-da-Silva, M., Gasparetti, A. L., Bezzera, R. M. N., Alberici, L. C., Vercesi, A. E., Saad, M. J. A., and Velloso, L. A. (2004) Cold-induced PGC-1{alpha} expression modulates muscle glucose uptake through an insulin receptor/Akt-independent, AMPK-dependent pathway. Am J Physiol Endocrinol Metab 287, E686-695
40. Schwer, B., Bunkenborg, J., Verdin, R. O., Andersen, J. S., and Verdin, E. (2006) Reversible lysine acetylation controls the activity of the mitochondrial enzyme acetyl-CoA synthetase 2. Proc Natl Acad Sci USA
41. Hallows, W. C., Lee, S., and Denu, J. M. (2006) Sirtuins deacetylate and activate mammalian acetyl-CoA synthetases. Proc Natl Acad Sci USA
42. Clapham, J. C., Arch, J. R., Chapman, H., Haynes, A., Lister, C., Moore, G. B., Piercy, V., Carter, S. A., Lehner, I., Smith, S. A., Beeley, L. J., Godden, R. J., Herrity, N., Skehel, M., Changani, K. K., Hockings, P. D., Reid, D. G., Squires, S. M., Hatcher, J., Trail, B., Latcham, J., Rastan, S., Harper, A. J., Cadenas, S., Buckingham, J. A., Brand, M. D., and Abuin, A. (2000) Mice overexpressing human uncoupling protein-3 in skeletal muscle are hyperphagic and lean. Nature 406, 415-418
43. Cline, G. W., Vidal-Puig, A. J., Dufour, S., Cadman, K. S., Lowell, B. B., and Shulman, G. I. (2001) In vivo effects of uncoupling protein-3 gene disruption on mitochondrial energy metabolism. J Biol Chem 276, 20240-20244
44. Thomson, D. M., Herway, S. T., Fillmore, N., Kim, H., Brown, J. D., Barrow, J. R., and Winder, W. W. (2007) AMP-Activated Protein Kinase Phosphorylates Transcription Factors of the CREB Family 10.1152/japplphysiol.00900.2007. J Appl Physiol, 104:429-438.
45. Handschin, C., Rhee, J., Lin, J., Tart, P. T., and Spiegelman, B. M. (2003) An autoregulatory loop controls peroxisome proliferator-activated receptor gamma coactivator 1alpha expression in muscle. Proc Natl Acad Sci USA 100, 7111-7116
46. Herzig, S., Long, F., Jhala, U. S., Hedrick, S., Quinn, R., Bauer, A., Rudolph, D., Schutz, G., Yoon, C., Puigserver, P., Spiegelman, B., and Montminy, M. (2001) CREB regulates hepatic gluconeogenesis through the coactivator PGC-1. Nature 413, 179-183
47. Halse, R., Fryer, L. G., McCormack, J. G., Carling, D., and Yeaman, S. J. (2003) Regulation of glycogen synthase by glucose and glycogen: a possible role for AMP-activated protein kinase. Diabetes 52, 9-15
48. Cammisotto, P. G., Londono, I., Gingras, D., and Bendayan, M. (2008) Control of glycogen synthase through ADIPOR1-AMPK pathway in renal distal tubules of normal and diabetic rats. Am J Physiol Renal Physiol 294, F881-889
49. Holmes, B. F., Kurth-Kraczek, E. J., and Winder, W. W. (1999) Chronic activation of 5′-AMP-activated protein kinase increases GLUT-4, hexokinase, and glycogen in muscle. J Appl Physiol 87, 1990-1995
50. Song, X. M., Fiedler, M., Galuska, D., Ryder, J. W., Fernstrom, M., Chibalin, A. V., Wallberg-Henriksson, H., and Zierath, J. R. (2002) 5-Aminoimidazole-4-carboxamide ribonucleoside treatment improves glucose homeostasis in insulin-resistant diabetic (ob/ob) mice. Diabetologla 45, 56-65
51. Barnes, B. R., Marklund, S., Steiler, T. L., Walter, M., Hjalm, G., Amarger, V., Mahlapuu, M., Leng, Y., Johansson, C., Galuska, D., Lindgren, K., Abrink, M., Stapleton, D., Zierath, J. R., and Andersson, L. (2004) The 5′-AMP-activated protein kinase gamma3 isoform has a key role in carbohydrate and lipid metabolism in glycolytic skeletal muscle. J Biol Chem 279, 38441-38447
52. Barre, L., Richardson, C., Hirshman, M. F., Brozinick, J., Fiering, S., Kemp, B. E., Goodyear, L. J., and Witters, L. A. (2007) Genetic model for the chronic activation of skeletal muscle AMP-activated protein kinase leads to glycogen accumulation. Am J Physiol Endocrinol Metab 292, E802-811
53. Costford, S. R., Kavaslar, N., Ahituv, N., Chaudhry, S. N., Schackwitz, W. S., Dent, R., Pennacchio, L. A., McPherson, R., and Harper, M. E. (2007) Gain-of-function R225W mutation in human AMPKgamma3 causing increased glycogen and decreased triglyceride in skeletal muscle. PLoS ONE 2, e903
54. Luptak, I., Shen, M., He, H., Hirshman, M. F., Musi, N., Goodyear, L. J., Yan, J., Wakimoto, H., Morita, H., Arad, M., Seidman, C. E., Seidman, J. G., Ingwall, J. S., Balschi, J. A., and Tian, R. (2007) Aberrant activation of AMP-activated protein kinase remodels metabolic network in favor of cardiac glycogen storage. J Clin Invest 117, 1432-1439
55. Barnes, B. R., Glund, S., Long, Y. C., Hjalm, G., Andersson, L., and Zierath, J. R. (2005) 5′-AMP-activated protein kinase regulates skeletal muscle glycogen content and ergogenics. Faseb J 19, 773-779
56. McCarty, M. F. (2004) Chronic activation of AMP-activated kinase as a strategy for slowing aging. Med Hypotheses 63, 334-339
57. Apfeld, J., O'Connor, G., McDonagh, T., DiStefano, P. S., and Curtis, R. (2004) The AMP-activated protein kinase AAK-2 links energy levels and insulin-like signals to lifespan in C. elegans. Genes Dev 18, 3004-3009
58. Dagon, Y., Avraham, Y., Magen, I., Gertler, A., Ben-Hur, T., and Berry, E. M. (2005) Nutritional status, cognition, and survival: a new role for leptin and AMP kinase. J Biol Chem 280, 42142-42148
59. Hou, X., Xu, S., Maitland-Toolan, K. A., Sato, K., Jiang, B., Ido, Y., Lan, F., Walsh, K., Wierzbicki, M., Verbeuren, T. J., Cohen, R. A., and Zang, M. (2008) SIRT1 Regulates Hepatocyte Lipid Metabolism through Activating AMP-activated Protein Kinase 10.1074/jbc.M802187200. J Biol. Chem. 283, 20015-20026
60. Baur, J. A., Pearson, K. J., Price, N. L., Jamieson, H. A., Lerin, C., Kalra, A., Prabhu, V. V., Allard, J. S., Lopez-Lluch, G., Lewis, K., Pistell, P. J., Poosala, S., Becker, K. G., Boss, O., Gwinn, D., Wang, M., Ramaswamy, S., Fishbein, K. W., Spencer, R. G., Lakatta, E. G., Le Couteur, D., Shaw, R. J., Navas, P., Puigserver, P., Ingram, D. K., de Cabo, R., and Sinclair, D. A. (2006) Resveratrol improves health and survival of mice on a high-calorie diet. Nature 444, 337-342
61. Gerhart-Hines, Z., Rodgers, J. T., Bare, O., Lerin, C., Kim, S. H., Mostoslavsky, R., Alt, F. W., Wu, Z., and Puigserver, P. (2007) Metabolic control of muscle mitochondrial function and fatty acid oxidation through SIRT1/PGC-1alpha. Embo J 26, 1913-1923
62. Bruce, C. R., Anderson, M. J., Carey, A. L., Newman, D. G., Bonen, A., Kriketos, A. D., Cooney, G. J., and Hawley, J. A. (2003) Muscle oxidative capacity is a better predictor of insulin sensitivity than lipid status. J Clin Endocrinol Metab 88, 5444-5451
63. Kelley, D. E., Goodpaster, B., Wing, R. R., and Simoneau, J. A. (1999) Skeletal muscle fatty acid metabolism in association with insulin resistance, obesity, and weight loss. Am J Physiol 277, E1130-1141
64. Lowell, B. B., and Shulman, G. I. (2005) Mitochondrial dysfunction and type 2 diabetes. Science 307, 384-387
65. Petersen, K. F., Dufour, S., Befroy, D., Garcia, R., and Shulman, G. I. (2004) Impaired mitochondrial activity in the insulin-resistant offspring of patients with type 2 diabetes. N Engl J Med 350, 664-671
66. Petersen, K. F., Befroy, D., Dufour, S., Dziura, J., Ariyan, C., Rothman, D. L., DiPietro, L., Cline, G. W., and Shulman, G. I. (2003) Mitochondrial dysfunction in the elderly: possible role in insulin resistance. Science 300, 1140-1142
67. Fridlyand, L. E., and Philipson, L. H. (2006) Reactive species and early manifestation of insulin resistance in type 2 diabetes. Diabetes Obes Metab 8, 136-145
68. Mootha, V. K., Lindgren, C. M., Eriksson, K. F., Subramanian, A., Sihag, S., Lehar, J., Puigserver, P., Carlsson, E., Ridderstrale, M., Laurila, E., Houstis, N., Daly, M. J., Patterson, N., Mesirov, J. P., Golub, T. R., Tamayo, P., Spiegelman, B., Lander, E. S., Hirschhorn, J. N., Altshuler, D., and Groop, L. C. (2003) PGC-1alpha-responsive genes involved in oxidative phosphorylation are coordinately downregulated in human diabetes. Nat Genet 34, 267-273
69. Patti, M. E., Butte, A. J., Crunkhorn, S., Cusi, K., Berria, R., Kashyap, S., Miyazaki, Y., Kohane, I., Costello, M., Saccone, R., Landaker, E. J., Goldfine, A. B., Mun, E., DeFronzo, R., Finlayson, J., Kahn, C. R., and Mandarino, L. J. (2003) Coordinated reduction of genes of oxidative metabolism in humans with insulin resistance and diabetes: Potential role of PGC1 and NRF1. Proc Natl Acad Sci US A 100, 8466-8471
70. Cui, L., Jeong, H., Borovecki, F., Parkhurst, C. N., Tanese, N., and Krainc, D. (2006) Transcriptional repression of PGC-1alpha by mutant huntingtin leads to mitochondrial dysfunction and neurodegeneration. Cell 127, 59-69
71. St-Pierre, J., Drori, S., Uldry, M., Silvaggi, J. M., Rhee, J., Jager, S., Handschin, C., Zheng, K., Lin, J., Yang, W., Simon, D. K., Bachoo, R., and Spiegelman, B. M. (2006) Suppression of reactive oxygen species and neurodegeneration by the PGC-1 transcriptional coactivators. Cell 127, 397-408

### Example 2

### Nutrient-Sensitive Mitochondrial NAD⁺ Levels Dictate Cell Survival

A major cause of cell death caused by genotoxic stress is thought to be due to the depletion of NAD⁺ from the nucleus and the cytoplasm. Here we show that NAD⁺ levels in mitochondria remain at physiological levels following genotoxic stress and can maintain cell viability even when nuclear and cytoplasmic pools of NAD⁺ are depleted. Rodents fasted for 48 hours show increased levels of the NAD⁺ biosynthetic enzyme Nampt and a concomitant increase in mitochondrial NAD⁺. Increased Nampt provides protection against cell death and requires an intact mitochondrial NAD⁺ salvage pathway as well as the mitochondrial NAD⁺ -dependent deacetylases SIRT3 and SIRT4. We discuss the relevance of these findings to understanding how nutrition modulates physiology and to the evolution of apoptosis.

Approximately 2 billion years ago, eukaryotes evolved by subsuming a bacterial antecedent of modern mitochondria (Barile et al., 1996; Gray et al., 1999). Mitochondria still retain a variety of molecules that dictate cell survival, which at one time may have been important for the survival of the bacterial proto-mitochondrion (James et al., 1998). Elucidation of these cell survival pathways is considered key to the development of new approaches to treating a variety of human diseases including cancer and neurodegeneration (Porcu and Chiarugi, 2005).

One of the major causes of cell death due to genotoxic stress is hyperactivation of the NAD⁺-dependent enzyme poly(ADP-ribose) polymerase-1 (PARP-1), which depletes nuclear and cytoplasmic NAD⁺ causing the translocation of apoptosis inducing factor (AIF) from the mitochondrial membrane to the nucleus (Burkle, 2005; Cipriani et al., 2005; van Wijk and Hageman, 2005; Yu et al., 2002). One recent study reported that a fraction of PARP-1 is localized in mitochondria, which has led to speculation about the potential for mitochondrial NAD⁺ to determine cell fate (Du et al., 2003). Yet little is known about mitochondrial NAD⁺ biosynthesis (Barile et al., 1996; Berger et al., 2005; Kun et al., 1975), what the actual concentration of NAD⁺ is in mitochondria (Di Lisa and Bernardi, 2006), whether mitochondrial NAD⁺ levels change in response to biological stress or diet, and what impact this has on cell survival and metabolism (Porcu and Chiarugi, 2005; Viswanathan et al., 2005).

The sirtuins are a conserved family of deacetylases and mono-ADP-ribosyltransferases that use NAD⁺ as a co-substrate (Guarente and Picard, 2005). These unusual enzymes, which bear virtually no sequence homology to Class I and II HDACs (Denu, 2005; Frye, 2000), have emerged as key regulators of cell survival and organismal longevity (Guarente and Picard, 2005). The founding member of the sirtuin family, Saccharomyces cerevisiae Sir2, is an NAD⁺-dependent histone deacetylase that mediates lifespan extension by mild stress and calorie restriction (CR) (Imai et al., 2000; Lin et al., 2000; Rogina and Helfand, 2004; Smith et al., 2000; Tanny et al., 1999). Mammals have seven sirtuins, SIRT1-7. SIRT1, a nuclear deacetylase, regulates a variety of functions including cell survival, glucose homeostasis, and fat metabolism (Guarente, 2005). There are three mitochondrial sirtuins, SIRT3-5. SIRT3 and SIRT4 were recently shown to regulate acetyl-CoA synthetase 2 (AceCS2) and glutamate dehydrogenase, respectively (Haigis et al., 2006; Hallows et al., 2006; Schwer et al., 2002), but little else is known about their biological functions.

Increased gene dosage or enhanced activity of Sir2 extends lifespan in S. cerevisiae, C. elegans and D. melanogaster (Anderson et al., 2003; Kaeberlein et al., 1999; Lin et al., 2000; Rogina and Helfand, 2004; Tissenbaum and Guarente, 2001; Wood et al., 2004). Yeast Sir2 is positively regulated by PNC1, a stress- and calorie-responsive longevity gene that catalyzes the first and rate-limiting step in NAD⁺ biosynthesis from nicotinamide (NAM) (Anderson et al., 2003; Gallo et al., 2004). Whether or not mammals possess a functional equivalent of the PNC1 gene is not known. Clearly, finding a mammalian equivalent of a gene that governs sirtuin activity and promotes longevity has many potential implications, including our understanding of how CR extends lifespan in mammals.

The search for a mammalian equivalent of PNC1 has been complicated by the fact that the synthesis of NAD⁺ from NAM is different in mammals than in simple eukaryotes (Brenner, 2005). While yeast, worms and flies require four steps to synthesize NAD⁺ from NAM, mammals require only two (Rongvaux et al., 2002). In yeast, the first step is catalyzed by Pnc1 and in mammals by the NAM phosphoribosyltransferase Nampt, also known as PBEF or visfatin (Fukuhara et al., 2004; Rongvaux et al., 2002; Samal et al., 1994). Recent studies have shown that overexpression of Nampt increases SIRT1 activity (Revollo et al., 2004) and can protect cells from death due to PARP overexpression (Pillai et al., 2005), which is consistent with the hypothesis that Nampt is a functional equivalent of Pnc1 in mammals.

In this paper, we identify NAMPT as a stress- and nutrient-responsive gene that boosts mitochondrial NAD⁺ levels. Mass spectrometric methods are used to accurately measure NAD⁺ concentrations within mammalian mitochondria and to show that NAMPT expression and mitochondrial NAD⁺ levels increase in vivo after fasting. Evidence is presented that increased mitochondrial NAD⁺ promotes cell survival during genotoxic stress and that protection is provided by the mitochondrial sirtuins SIRT3 and SIRT4. These data show that mitochondrial NAD⁺ is a major determinant of apoptosis and shed new light on the influence of diet on organ physiology and disease.

### Methods

### Cell Culture

Human embryonic kidney (HEK293) and human fibrosarcoma HT1080 cell lines were obtained from ATCC and grown in complete DMEM medium with 10% FBS and 100 μg/ml penicillin/streptomycin. To generate Nampt overexpression or empty vector stable clones, hNAMPT/pcDNA or pcDNA empty vector were transfected into HEK293 or HT1080 cells and selected with 0.5-1.0 mg/ml geneticin. To generate Nampt knockdown cells, siRNA/NAMPT/pMSCV or pMSCV empty vector were transfected into HT1080 cells, and stable clones were selected with 500 ng/ml puromycin. Primary neonatal rat cardiomyocytes were prepared as described previously (Matsui et al., 1999). Isolated cardiomyocytes were grown in complete medium (RPMI 1640, 5% fetal calf serum, 10% horse serum) to 80% confluence before cells were subjected to hypoxia and/or serum starvation. For hypoxia, medium was changed to RPMI 1640 with or without serum saturated with 95% N₂/5% CO₂ and then cells were placed in a 37° C. airtight box saturated with 95% N₂/5% CO₂ for 18 hours. O₂ concentrations were <0.1% (Ohmeda oxygen monitor, type 5120). To serum starve HT1080, cells were grown in DMEM medium containing no serum. Cells were harvested after 26 h of serum starvation and Nampt expression was analyzed by Western blotting.

### Drug Treatments and Cell Death Assays

HEK293 and HT1080 cells at less than 85% confluency were treated with MMS (1.2 mM) for 6-8.5 h and 8-17 h, respectively. Transiently transfected cells were washed 3 times with PBS to remove residual transfection reagents before treating with MMS. HEK293 cells and HT1080 cells were exposed to etoposide for 72 h or 46 h, at 120 or 40 μM, respectively. HT1080 cells were exposed to camptothecin at 14 μM for 23 h. After drug treatments, cells were harvested by trypsinization, pelleted by centrifugation and resuspended in PBS containing 3% FBS. Cell death was analyzed by FACS using propidium iodide (PI) staining. Cells transiently-transfected with mNAMPT/MSCV, SIRT1-7 siRNA, Nmnat-3, or hMFT siRNA oligos and co-transfected with FAM-labeled scrambled siRNA oligos, percent cell death was determined by as the proportion of PUGFP or PIFAM positive cells versus the total number of cells with green fluorescence.

### Assay for Aceylated AceCS2

Control and Nampt overexpressing HEK293 cells were transfected with control vector or AceCS2-HA. 48 hrs post transfections, cells were washed and lysed in IP lysis buffer [50 mM Tris-HCl, pH 7.5, 150 mM NaCl, 0.5 mM EDTA, 0.5% NP-50, 400 nM TSA, 5 mM Nicotinamide, and protease inhibitors (Compelete, Roche)] for 30 minutes at 4 degC. Lysates where cleared and subjected to immunoprecipitation with anti-HA affinity gel (Roche) for 2 hrs at 4° C.

Immunoprecipitations were washed 3 times for 15 min each in lysis buffer and resuspended in 1×SDS-PAGE buffer and analyzed by western blotting.

### Cell Fractionation and Drug Treatment of Mitochondria

Unless otherwise stated, fractionation of cultured cells was performed using a differential centrifugation and sucrose gradient procedure with slight modifications (Schwer et al., 2002). For drug treatment of mitochondria, fresh mitochondria were obtained from livers excised from fed young male rats. Half of the liver homogenate was used for mitochondrial isolation using a commercially available kit as described by manufacturer's instructions, designated Protocol 1 (Pierce Mitochondrial Isolation kit, Rockford, Ill.). The other half of the original homogenate was subjected to a differential centrifugation protocol to isolate mitochondria (Protocol 2). Mitochondria (500 μl) were added to a 48-well plate and treated with methylmethane sulfonate (MMS) at 1:1000 dilution and/or FK866 (10 nM) for 30 min at 37° C. Suspensions were spun down for 1 min at 14,000 rpm at 4° C. and pellets were stored at −80° C. prior to NAD⁺ analysis by HPLC-MALDI-MS. Detailed protocols are described in Supplemental Information.

### HPLC-MALDI-MS Determination of NAD⁺

NAD⁺ was determined by as described previously (Sauve and Schramm, 2003), with the following modifications. HEK293 or HT1080 cells were harvested by trypsinization and counted by haemocytometer. NAD⁺ was extracted from whole cells or mitochondrial pellets by adding 10% perchloric acid and sonicating for 5 min on ice. The reference standard ¹⁸O-NAD⁺ (typically 613.4 μmol) was then added to the sample. After mixing well, samples were centrifuged for 3 min then neutralized with NaOH. NAD⁺ in the supernatant (100 μL sample) was separated from other cellular components by HPLC. NAD⁺ peaks were collected according to the standards' retention time and dried with a lyophilizor. MALDI-MS was used to determine the peak ratio between the positively charged isotopically distinct ions, and the intensities of the ¹⁶O- and ¹⁸O-peaks were ratioed (664/666) to obtain ¹⁶O NAD⁺/¹⁸O NAD⁺ in the sample. Standards containing only ¹⁶O and ¹⁸O NAD⁺ (600 μmol each) were also run to determine corrections for isotopic purity and to provide calibration of the procedure. The volume of mitochondrial pellets was calculated by considering the density of the mitochondria pellets to be 1.0 μL/mg. Total cellular NAD⁺ concentrations were calculated by dividing the quantity of NAD⁺ per cell by the mean cell volume (MCV) as measured by a Coulter counter, which for HT1080 was 2183.95 μL and for HEK293 was 1691.04 μL.

### Animal Experiments

To assess Nampt expression in vivo, Sprague-Dawley rats (120-150 g) were obtained from Charles River Laboratories and randomly assigned to control and experimental groups of 4 animals. The control group was fed ad libitum with a 78% sucrose diet prepared by Research Diets Inc. and the experimental group was fasted for 48 hours before being sacrificed. Liver tissue for RNA extraction was stored in RNAlater reagent (Qiagen). All other samples were frozen in liquid nitrogen and stored at −80° C. until use. To assess mitochondrial Nampt and NAD⁺ levels, Male Fischer-344 (F344) rats were bred and reared in a vivarium at the Gerontology Research Center (GRC, Baltimore, Md.). From weaning (2 w), the rats were housed individually in standard plastic cages with beta chip wood bedding. Control animals were fed a NIH-31 standard diet ad libitum (AL). The procedures for preparation of mitochondria from liver are described in Supplementary Information.

### Reagents and Antibodies

The rabbit polyclonal anti-Nampt antibody was generated by immunizing rabbits with purified human Nampt protein expressed in E. coli. Mouse anti-Nampt mAb (14A5) was generously supplied by Anthony Rongvaux & Oberdan Leo (Laboratoire de Physiologic Animale, Universite Libre de Bruxelles, Gosselies, Belgium). Anti-SIRT3 antibody was generated by immunizing rabbits with synthesized peptide corresponding to the last 15 amino acid residues of the C-terminus of the human SIRT3 protein. Anti-SIRT2, and anti-acetylated-AceCS2 antibodies were gifts from Eric Verdin, UCSF. Anti-SIRT5 antibody was purchased from BIOMOL. Anti-SIRT7 antibody was generously supplied by Izumi Horikawa (Laboratory of Biosystems and Cancer, NIH). Anti-V5 antibody was bought from Invitrogen. Anti-cleaved caspase-3 antibody was from Cell Signaling, the rabbit polyclonal mouse anti-Sir2α antibody from Upstate, anti-AIF and anti-actin antibodies from Santa Cruz Biotechnology, anti-Hsp90 and anti-MnSOD from Stressgen, anti-CoxIV and anti-GAPDH from Abcam, anti-EZH2 from BD Biosciences. MMS and sirtinol were purchased from Sigma. DPQ (3,4-dihydro-5-[4-(1-piperidinyl)butoxy]-1(2H)-isoquinolinone), camptothecin, etoposide were bought from EMD. FK866 ((E)-[4-(l-Benzoylpiperidin-4-yl)butyl]-3-pyridin-3-yl)acrylamide) was kindly supplied by RTI (Research Triangle Institute). EX-527 (6-Chloro-2,3,4,9-tetrahyro-1H-carbazole-1-carboxylate) was a gift from Jill Milne (Sirtris Pharmaceuticals, Cambridge, Mass.). FuGENE6 transfection reagent was from Roche, lipofection 2000 from Invitrogen, and small interfering RNA (siRNA) oligos targeted to human SIRT1-7, hNmnat-3 or hMFT mRNA from Dharmacon.

### DNA Constructs

Human NAMPT/pcDNA was a gift from Gillian Bryant-Greenwood (University of Hawaii). Plasmids of mNAMPT/MSCV and the corresponding empty vector were from Anthony Rongvaux & Oberdan Leo (Laboratoire de Physiologie Animale, Universite Libre de Bruxelles, Gosselies, Belgium). Human SIRT1-7-flag/pcDNA constructs, AceCS2-HA and the corresponding control vectors were kindly supplied by Eric Verdin (University of California, San Francisco). pMSCV-U6 was a gift from Yang Shi (Harvard Medical School, Boston), and was used to construct the viral plasmid for expressing siRNA against the Nampt sequence GGTGAAGATCTAAGACATTTA (SEQ ID NO:9) (siRNA/NAMPT/pMSCV).

### RNA Procedures

Total RNA from liver tissue or HEK293 cells transiently-transfected with scrambled siRNA or siRNAi oligos targeting to SIRT4, SIRT6, hMFT and hNmant-3 was extracted using Trizol (Invitrogen), mRNA was reverse transcribed using superscript Ill (Invitrogen) and oligo dT or random hexamer primers for rat liver and HEK293 samples, respectively. cDNA generated by reverse transcription was used as a template for real-time PCR using a LightCycler and FastStart DNA Master SYBR Green I (Roche Molecular Biochemicals). Relative copy numbers of NAMPT, SIRT4, SIRT6, Nmant-3 and hMFT mRNA were determined by comparison to β-actin. Primer sets used in quantitative real-time PCR reactions were:

(SEQ ID NO: 10)
AAATCCGCTCGACACTGTCCTGAA
and
(SEQ ID NO: 11)
TTGGGATCAGCAACTGGGTCCTTA for rat NAMPT;
(SEQ ID NO: 12)
TTCCTCCCTGGAGAAGAGCTATGA
and
(SEQ ID NO: 13)
TACTCCTGCTTGCTGATCCACATC for rat β-actin;
(SEQ ID NO: 14)
ACTTCGTAGGCTGGCCTCAATTCT
and
(SEQ ID NO: 15)
ACATCTGGTTTCAGATGGCCTCCA for hSIRT4;
(SEQ ID NO: 16)
AGAGCTCCACGGGAACATGTTTGT
and
(SEQ ID NO: 17)
ATGTACCCAGCGTGATGGACAGGT for hSIRT6;
(SEQ ID NO: 18)
TGGCCAGAGATCACCTACACCAAA
and
(SEQ ID NO: 19)
TGATGATGCCTCAGCACCTTCACT
for hNmant-3;
(SEQ ID NO: 20)
AGGGATTTGTTCCTGGGCTGTTTG
and
(SEQ ID NO: 21)
AAATCCACCGACGCCTTCTTTCCT for hMFT;
(SEQ ID NO: 22)
TTCTACAATGAGCTGCGTGTGGCT
and
(SEQ ID NO: 23)
TTAATGTCACGCACGATTTCCCGC for human β-actin.

### Cell Fractionation

Unless otherwise stated, fractionation of cultured cells was performed using a differential centrifugation and sucrose gradient procedure with slight modifications (Schwer et al., 2002). Supernatants from differential centrifugation were subjected to ultracentrifugation to precipitate the light membranes and collected as cytosolic S-100 fractions. The pellets from the differential centrifugation were washed with butter 3 times and used as nuclear fractions. Aliquots of isolated mitochondrial samples were lysed and mitochondrial protein concentrations were determined using a Bradford assay. Protein extracts (40 μg) from S-100, mitochondrial fractions and nuclear fractions were separated by SDS-PAGE and probed for Nampt, Hsp90 and CoxIV by Western blotting. Nuclei were isolated from HEK293 cells by using a protocol from Dr. Angus Lamond's lab (School of Life Sciences, Wellcome Trust Biocentre University of Dundee, UK), which is a variation of a previously published procedure (Busch et al., 1963). Briefly, cells were harvested, washed in cold PBS and resuspended in 5 ml hypotonic buffer A (10 mM HEPES, pH 7.9, 10 mM KCl, 1.5 mM MgCl₂, 0.5 mM DTT) and incubated on ice for 5 min. The cell suspension was transferred to a pre-cooled 7 ml Dounce tissue homogenizer and homogenized until >90% of the cells had lysed. Nuclei were pelleted by centrifugation at 218×g. Nuclei were resuspended in 3 ml of S1 solution (0.25 M sucrose, 10 mM MgCl₂) and layered the suspension over 3 ml of S2 solution (0.35 M sucrose, 0.5 mM MgCl₂). Ultra-pure nuclear pellets were obtained by centrifugation at 1430×g for 5 min at 4° C.

Cell fractionation from tissue was performed the following way unless mitochondria were used for drug treatments. Groups of five animals were sacrificed between 9 and 11 a.m. after ad libitum feeding or after a 48 h fast. Animals were sacrificed by decapitation. The liver was removed immediately and placed on ice-cold isolation buffer prior to mitochondrial isolation. Each dissected liver was placed in a glass dounce homogenizer containing four times the volume of isolation buffer with 1 mM EGTA (215 mM mannitol, 75 mM sucrose, 0.1% BSA, 20 mM HEPES, 1 mM EGTA and pH is adjusted to 7.2 with KOH). The tissue was homogenized and mitochondria were isolated by differential centrifugation. Briefly, the homogenate was centrifuged at 1300×g for 3 min in a microcentrifuge at 4° C. and the supernatant was transferred to new tubes. The loose pellet was resuspended in isolation buffer with EGTA and was centrifuged again at 1300×g for 3 min. The resulting supernatant was transferred to new microcentrifuge tubes and topped off with isolation buffer with EGTA and centrifuged at 13,000×g for 10 min. The supernatant was discarded and the pellet was resuspended in 500 μl of isolation buffer with EGTA. The sample was further purified by percoll density gradient centrifugation as previously described (Nukala et al., 2006). The tubes were topped off with isolation buffer without EGTA (215 mM mannitol, 75 mM sucrose, 0.1% BSA, 20 mM HEPES, pH adjusted to 7.2 with KOH) and centrifuged at 13,000×g for 10 min at 4° C. The supernatants were carefully removed and the pellets were resuspended in 500 μl of isolation buffer without EGTA. The tubes were topped off with isolation buffer without EGTA and centrifuged at 10,000×g for 5 min at 4° C. to yield a tighter pellet. The final mitochondrial pellet was resuspended in isolation buffer without EGTA. The protein concentration was determined using the BCA protein assay kit measuring absorbance at 562 nm with a Victor 3 μlate reader. These mitochondrial samples were subjected to Western analysis and NAD⁺ determination.

For mitochondrial drug treatments, fresh mitochondria were obtained from livers excised from fed young male rats. Livers were minced using razor blades and disrupted with a Dounce homogenizer using ˜10 strokes in 30 ml of PBS. Half of the homogenate was used for mitochondrial isolation using a commercially available kit as described by manufacturer's instructions, designated Protocol 1 (Pierce Mitochondrial Isolation kit, Rockford, Ill.). Briefly, homogenates were transferred to 1.5 ml tubes and centrifuged at 1000×g (3000 rpm) for 3 min at 4° C. The supernatant was discarded and the pellet resuspended in 600 μl of Reagent A containing 4 mg/ml BSA. Samples were vortexed at medium speed for 5 sec and incubated on ice for 2 min. After adding 10 μl of Reagent B, samples were vortexed at maximum speed for 5 sec and then incubated on ice for 5 min, vortexing again every min. Reagent C (600 μl) was added and mixed by inversion. Samples were centrifuged at 700 g (2600 rpm) for 10 min at 4° C. The supernatant was transferred to a new tube and centrifuged at 3000 g (5300 rpm) for 15 min at 4° C. The pellet was resuspended in wash buffer and centrifuged at 12,000 g (10,700 rpm) for 5 min and the supernatant was discarded.

The other half of the original homogenate was subjected to a differential centrifugation protocol to isolate mitochondria (Protocol 2). The homogenate was added to two 50 ml ultracentrifuge tubes and isolation buffer was added to make a final volume of 30 ml. Solutions were centrifuged for 5 min at 700×g at 4° C. Supernatant was transferred to new centrifuge tubes. The process was repeated until there was no visible pellet. The supernatant was then spun for 15′ at 6,000 g at 4° C. The pellet containing mitochondria was resuspended in 20 ml isolation buffer with PMSF and respun two more times. After isolation, mitochondrial pellets were resuspended in 6 ml respiration buffer (215 mM mannitol, 75 mM sucrose, 2% BSA, 2 mM MgCl₂, 2.5 mM KH₂PO₄, and 20 mM HEPES, pH 7.4).

### Results

### NAMPT Expression is Induced by Cell Stress and Nutrient Restriction

Others and we have speculated that mammalian Nampt is the functional equivalent of yeast Pnc1 (Anderson et al., 2002; Anderson et al., 2003; Bitterman et al., 2003; Revollo et al., 2004; Yang et al., 2006). This idea was based on the fact that both Pnc1 and Nampt catalyze the first and rate-limiting step in NAD⁺ biosynthesis from nicotinamide (NAM) (Pillai et al., 2005; Revollo et al., 2004; Rongvaux et al., 2002).

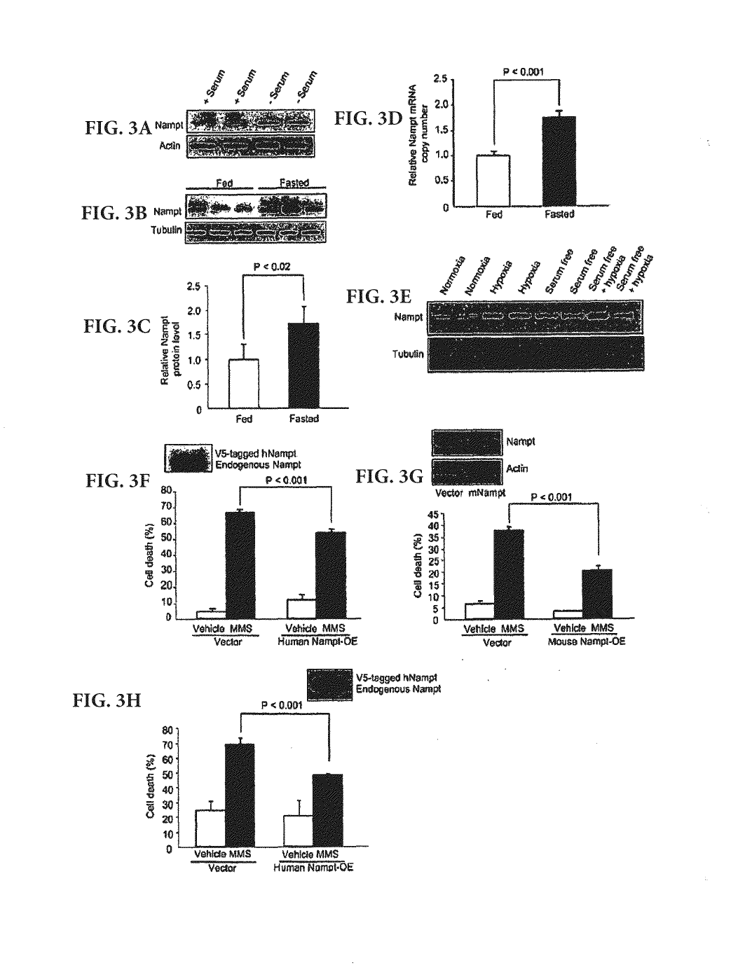

We reasoned that if NAMPT is analogous to the yeast PNC1 gene, then its expression might also be induced by cell stress and nutrient restriction. We found that human fibrosarcoma HT1080 cells cultured in serum-free conditions had ˜1.5- to 2-fold the levels of Nampt relative to controls (FIG. 3A). A similar increase in Nampt at both the mRNA and protein level was observed in the livers of rats that were subjected to a 48 h fast (FIG. 3B-D). Primary cardiomyocytes exposed to hypoxia or serum free media also had ˜2-fold higher levels of Nampt (FIG. 3E). Similar results were observed in primary mouse embryonic fibroblasts (MEFs) grown in serum-free medium (data not shown). Thus, NAMPT is a stress- and nutrient-responsive gene, similar in this regard to the yeast PNCJ gene.

### Nampt Protects Against Cell Death Due to Genotoxic Stress

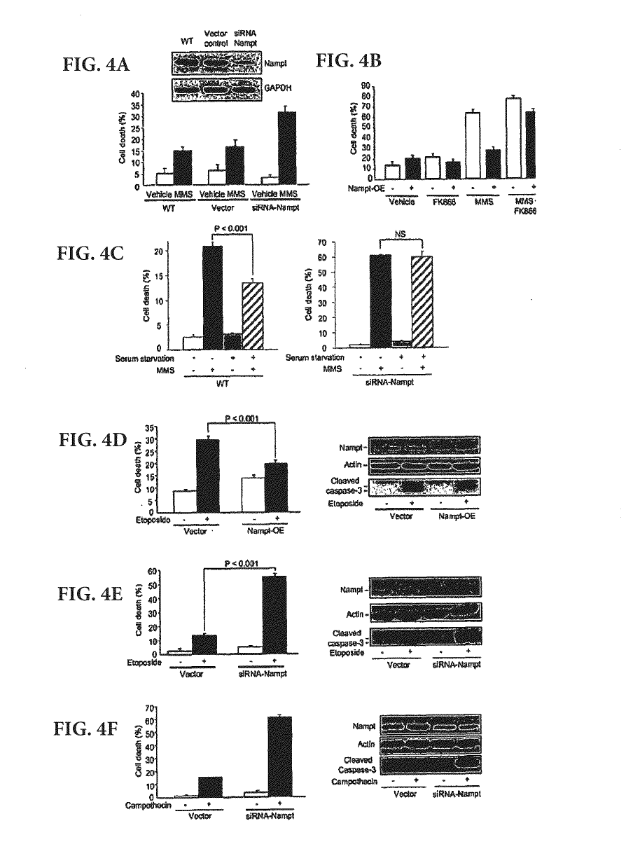

To mimic the upregulation of Nampt and to test what effect this has on stress resistance, we generated human Nampt-overexpressing stable cell lines from HT1080 fibrosarcoma and HEK293 embryonic kidney cells, and selected lines that expressed 1.5 to 2 times the level of Nampt relative to vector controls (FIG. 3F,H). A similar increase in Nampt expression was obtained by transiently transfecting HT1080 cells with a mouse Nampt expression construct (FIG. 3G). Stable Nampt knockdown cell lines were also generated using siRNA (FIG. 4A).

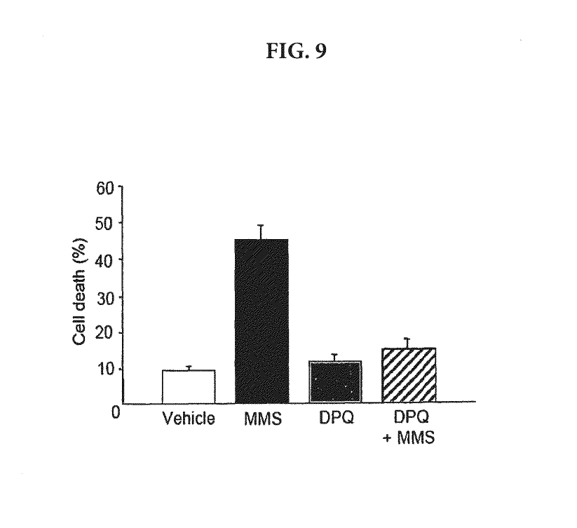

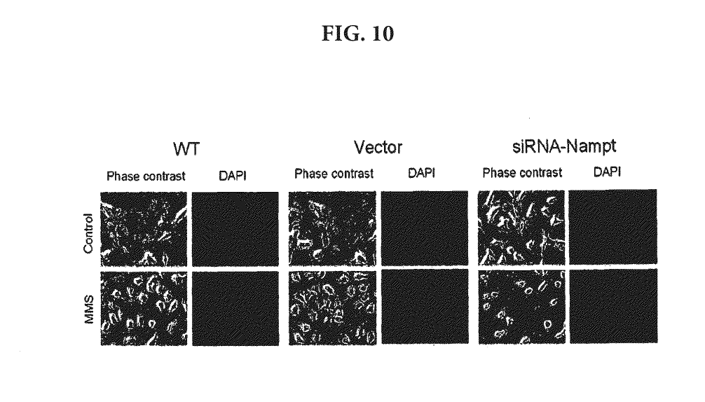

Cell lines were then assayed for their sensitivity to methylmethane sulfonate (MMS), a DNA alkylating agent that is known to hyperactivate PARP-1 (Horton et al., 2005). MMS treatment resulted in the death of about half the vector control cells and the extent of cell death was greatly reduced by the PARP inhibitor, DPQ (FIG. 9), indicating that PARP-hyperactivation and depletion of NAD⁺ were the primary mode of death under these conditions. Although Nampt-overexpressing cells had only slightly higher levels of Nampt protein, they were substantially more resistant to MMS than controls (FIG. 3F-H). Conversely, cells with lower levels of Nampt were more sensitive to MMS (FIG. 4A and FIG. 10). A potent Nampt catalytic inhibitor, FK866, which binds in the active site (Drevs et al., 2003), prevented cell protection by Nampt overexpression (FIG. 4B), indicating that Nampt activity is required for cell protection.

Given that Nampt levels increase in cells grown in serum free media, we wondered whether cells in serum free media are more resistant to MMS and, if so, whether their resistance is mediated by Nampt. As shown in FIG. 4C, cells that were serum starved were more resistant to MMS and this resistance was entirely Nampt-dependent.

We also tested whether Nampt provides resistance to cell death from other types of DNA damage. Etoposide is a cancer chemotherapeutic agent that inhibits topoisomerase II, resulting in numerous double-stranded DNA breaks that trigger apoptosis. Nampt-overexpressing cells were more resistant to etoposide and had reduced levels of cleaved caspase 3, a marker of apoptosis (FIG. 4D). Conversely, cells with reduced levels of Nampt were more sensitive to etoposide and had increased levels of cleaved caspase 3 (FIG. 4E). The Nampt knockdown cells were also more sensitive to camptothecin, a topoisomerase I inhibitor (FIG. 4F), demonstrating that the ability of Nampt to protect from cell death is not specific to MMS.

### Nampt-Mediated Cell Protection Requires Mitochondrial SIRT3 and SIRT4

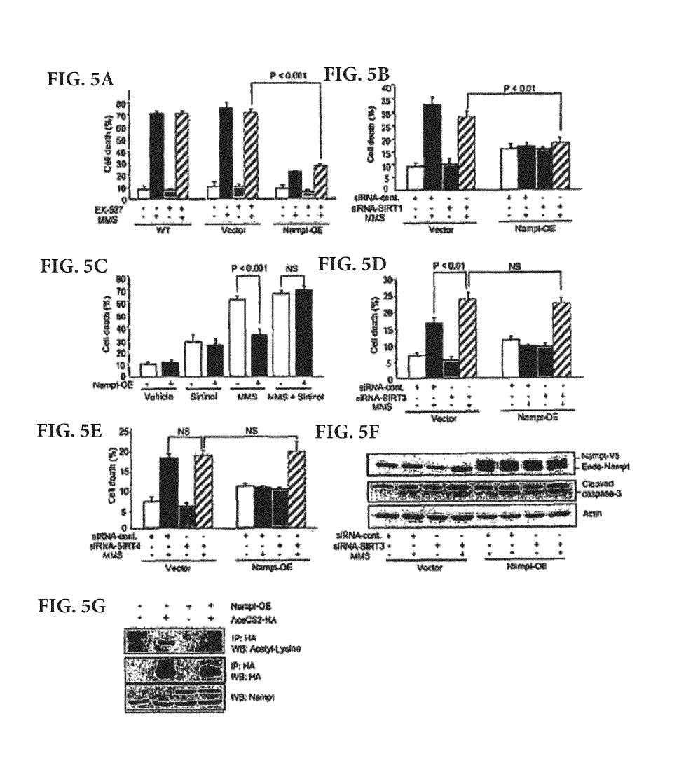

In light of a recent study showing that the ability of Nampt to protect against PARP-1 overexpression was SIRT1-mediated (Pillai et al., 2005), we expected that protection from MMS would be SIRT1-dependent. But neither EX-527, a SIRT1-specific inhibitor (Solomon et al., 2006), nor siRNA-mediated knockdown of SIRT1, had a significant effect on Nampt-mediated survival (FIGS. 5A, B, and 11A). Curiously though, treatment of cells with the pan-sirtuin inhibitor, sirtinol, did block the ability of Nampt overexpression to protect cells (FIG. 5C), which raised the possibility that survival might be mediated by another sirtuin.

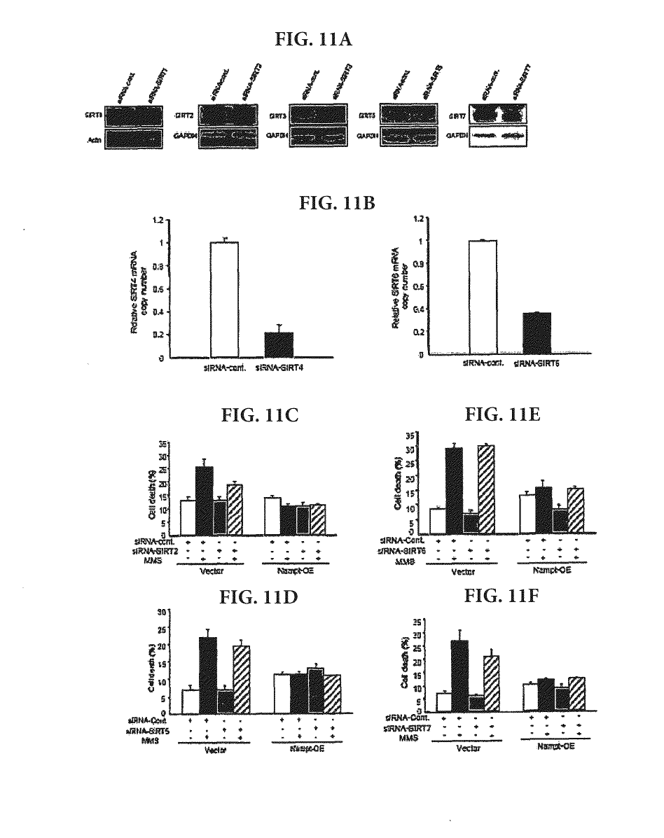

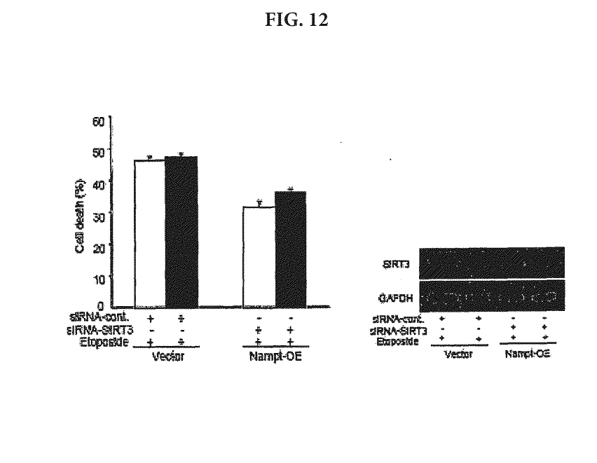

To test this idea, we knocked-down each of the remaining sirtuins, SIRT2-7, using siRNA (FIG. 11A-B) and scored survival after MMS treatment. Interestingly, the mitochondrial sirtuins, SIRT3 and SIRT4, but not the other sirtuins, were required for the ability of Nampt to protect against MMS-induced cell death (FIGS. 5D, E and 11C-F). In addition, knockdown of SIRT3 sensitized wild-type cells to MMS and increased the relative abundance of cleaved caspase 3 (FIGS. 5D, F and data not shown). This effect appears to be relatively specific to MMS-induced cell death because there was no appreciable effect on sensitivity to etoposide (FIG. 12).

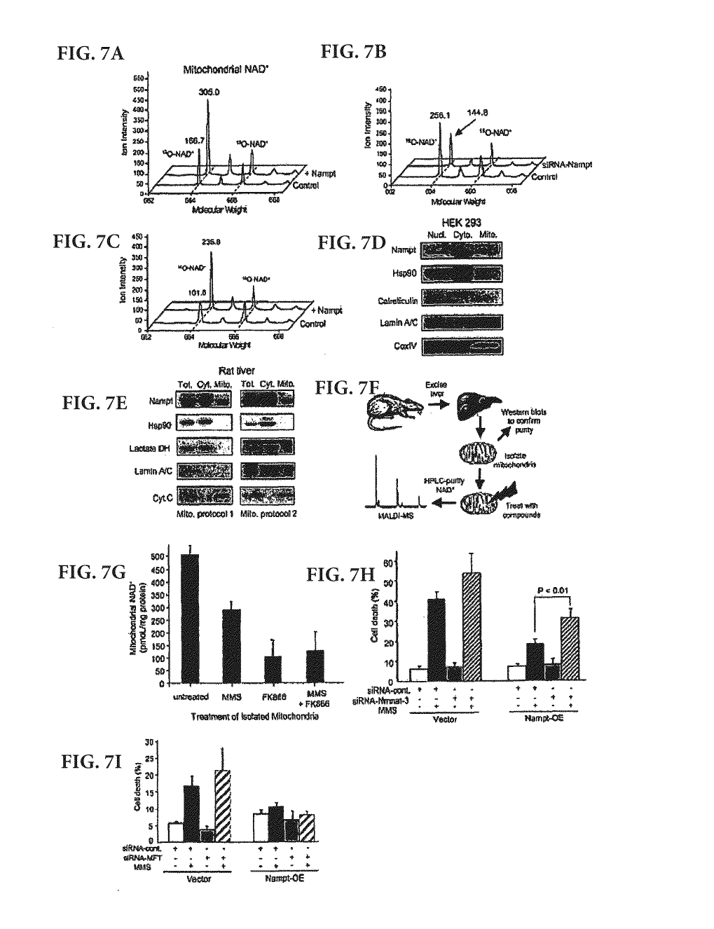

These data indicated that overexpression of Nampt might protect cells by increasing SIRT3 activity. We tested this by monitoring the acetylation status of AceCS2, a substrate of SIRT3. As shown in FIG. 5G, Nampt overexpression markedly reduced acetylation level of AceCS2, indicating that Nampt increases SIRT3 activity. Knockdown of SIRT4, on the other hand, had no significant effect on the survival of wild type cells (FIG. 5E and data not shown). There was no significant increase in the survival of cells overexpressing SIRT3 or SIRT4, or in combination (data not shown), suggesting that in the absence of additional Nampt, MMS causes NAD⁺ levels to drop below the concentration at which these NAD⁺ -dependent enzymes can function, a possibility we tested below (see FIG. 7).

Nampt does not Prevent Depletion of Total NAD+

It is generally recognized that genotoxic stress kills cells by depleting nuclear and cytosolic NAD+ pools (Burkle, 2005; Porcu and Chiarugi, 2005). To test whether Nampt protected against NAD+ depletion, we measured NAD+ concentrations in HEK293 cells after exposure to MMS using an quantitative HPLC-mass spectrometry method (HPLC-MALDI-MS) (Sauve et al., 2005). We did not attempt to measure nicotinamide levels in mitochondria because, unlike NAD+, it diffuses through membranes (van Roermund et al., 1995).

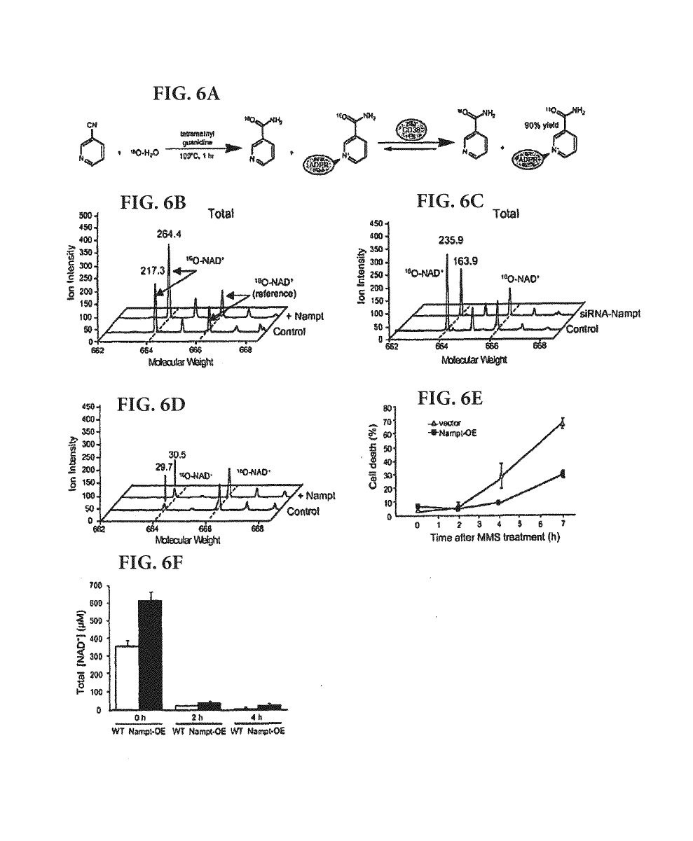

An internal NAD+ reference, ¹⁸O-NAM, was synthesized from 3-cyanopyridine, then enzymatically converted to 18O-NAD+ using a recombinant NAD+ glycohydrolase, CD38 (FIG. 6A). The key advantage of the HPLC-MALDI-MS technique is that extracts are spiked with an isotopically-labeled NAD+ internal reference standard so that losses of NAD+ during purification do not affect the final result. After addition of the labeled standard, the NAD+ in the cell extracts was HPLC purified and analyzed by mass spectrometry, which gave two peaks that corresponded to the two isotopomer molecular ions (FIG. 6B). The higher molecular weight species, 18O-NAD+, was used to determine the quantity of the lower weight endogenous species, 16O-NAD+.

We estimate that the total NAD⁺ concentration in HEK293 cells is 365+/−30.2 μM, which is very close to a recent estimate for mouse erythrocytes using HPLC-MS-electrospray ionization (368 μM) (Yamada et al., 2006). Interestingly, Nampt-overexpressing cells had approximately twice the total NAD+ concentration of vector control cells and, conversely, Nampt knockdown cells had approximately half (FIGS. 6B and C). These data are consistent with other studies showing that Nampt catalyzes a rate-limiting step in nucleo-cytoplasmic NAD+ biosynthesis (Pillai et al., 2005; Revollo et al., 2004).

Surprisingly, Nampt overexpression did not appreciably affect MMS-mediated depletion of NAD+ in total cell extracts (FIG. 6D). To ensure that we had not simply chosen an inappropriate time point, total NAD+ levels were measured at time points just prior to, and during which, cell protection by Nampt was observed (2 and 4 h) (FIG. 6E). Again, concentrations of total NAD+ became critically low (total [NAD+]<100 μM), irrespective of Nampt levels (FIG. 6F).

### Nampt-Regulated Mitochondrial NAD+ Levels Dictate Cell Survival

These data raised an intriguing question: How does Nampt protect cells from genotoxic stress if not by maintaining total NAD+ levels? An important clue came from our observation that the mitochondrial sirtuins SIRT3 and SIRT4 are required for Nampt to increase cell survival. The cell lines we were using, HT1080 and HEK293, possess relatively few mitochondria compared to highly metabolically-active cells such as hepatocytes and myocytes (Di Lisa and Bernardi, 2006), and hence mitochondria contribute only a minor component of the total NAD+ pool of these cells. It was plausible that the NAD+ remaining in the MMS-treated cells was primarily mitochondrial and that the increased survival in Nampt overexpressors was a consequence of increased NAD+ levels in mitochondria.

Surprisingly little is known about the precise concentration of NAD+ in mammalian mitochondria or whether changes in mitochondrial NAD+ levels impact cell survival (Porcu and Chiarugi, 2005; Viswanathan et al., 2005). This lack of knowledge appears to stem, in part, from the lack of a robust and accurate method to measure mitochondrial NAD+ concentrations. Indeed, only a few studies have attempted to measure mitochondrial NAD+ levels by any means. Using enzymatic assays, it has not been possible to accurately determine actual concentrations of NAD+ within the organelle. Levels are typically expressed not in molarity but as a nmol NAD+/mg mitochondrial protein (Di Lisa and Bernardi, 2006; Noack et al., 1992; Tobin et al., 1980)

We began by measuring actual mitochondrial NAD+ concentrations using an adaptation of the MALDI-MS technique described above (see Materials and Methods). We estimate the concentration of NAD+ in mitochondria of HEK293 cells is 245.6 μM, corresponding to 2053 μmol NAD+/mg mitochondrial protein. Consistent with our hypothesis, cells with additional Nampt had approximately double the concentration of NAD+ in mitochondria (FIG. 7A) and there was a corresponding decrease in mitochondrial NAD+ in cells in which Nampt was knocked down (FIG. 7B). Importantly, Nampt did not alter mitochondrial size or number, and losses of NAD+ during purification post-addition of the NAD+ reference would not have affected the result.

Next, we determined the effect of Nampt on mitochondrial NAD+ after MMS treatment. Strikingly, Nampt-overexpressing cells had more than double the concentration of mitochondrial NAD+ relative to MMS-treated wild type controls (FIG. 7C). In fact, the concentration of mitochondrial NAD+ in Nampt overexpressing cells was higher during MMS exposure than wild type cells that were untreated. Thus, mitochondria of Nampt-overexpressing cells retain physiological levels of NAD+ after MMS treatment, even if the rest of the cell is depleted of NAD+.

How do Mitochondria Maintain NAD+ and Protect Cells from Apoptosis?

Next, we wondered how mitochondria maintain such high NAD+ levels during genotoxic stress. There were two plausible explanations. Mitochondria might possess an endogenous NAD+ biosynthetic pathway and/or they might import NAD+ from the cytosol. We first tested the hypothesis that Nampt is localized to mitochondria and participates in mitochondrial NAD+ synthesis. There is already good evidence for an NAD+ salvage pathway in mitochondria. Two studies showed that an NAD+ precursor can be converted to NAD+ when added to mitochondrial preparations (Barile et al., 1996; Kun et al., 1975) and, more recently, an enzyme immediately downstream of Nampt in the NAD+ salvage pathway, Nmnat3 (for NAM mononucleotide adenylyltransferase), was shown to be exclusively mitochondrial (Berger et al., 2005).

To explore whether mitochondria contain Nampt, we isolated highly pure mitochondrial fractions from either HEK293 cells (FIG. 7D) or from fresh rat livers, in the latter case using two different mitochondrial isolation methods (FIG. 7E). The purity of the fractions was assessed by probing for cytoplasmic markers (Hsp90, calreticulin, lactate dehydrogenase), mitochondrial matrix markers (CoxIV or cytochrome C), and a nuclear membrane protein (lamin A/C). The enrichment of mitochondrial markers in the mitochondrial fractions and the absence of cytoplasmic and nuclear proteins in these fractions demonstrated that the mitochondrial preparations were highly pure. Nampt was observed in cytoplasmic and nuclear fractions, consistent with other reports (Kitani et al., 2003; Revollo et al., 2004; Rongvaux et al., 2002). In all of the mitochondrial preparations, from HEK293 cells and from liver tissue, Nampt was clearly detected. Nampt was also present in highly pure mitochondrial preparations from human lymphoblasts, HepG2 hepatocarcinoma cells and HeLa cells (not shown).

To add weight to these findings, we performed functional assays for Nampt activity in mitochondria. Specifically, we tested whether interfering with Nampt activity in mitochondria affects mitochondrial NAD+ levels and whether blocking mitochondrial NAD+ biosynthesis reduced the ability of Nampt to protect from genotoxic stress. We reasoned that if Nampt activity is required to maintain mitochondrial NAD+, then inhibiting Nampt in isolated mitochondria should reduce mitochondrial NAD+ levels. On the other hand, if mitochondrial NAD+ is derived from the cytoplasm, then inhibiting Nampt activity should have no effect on the levels of NAD+ in purified mitochondria.

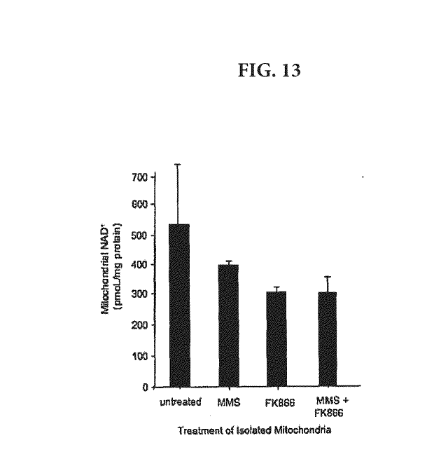

Mitochondria from fresh rat livers were purified and treated in vitro with MMS or FK866 or in combination (FIG. 7F). NAD+ content in the mitochondria was then determined using the HPLC/mass spectrometry method utilized above for whole cells. Treatment of isolated mitochondria with MMS reduced mitochondrial NAD+ levels by ˜2-fold (FIG. 7G), similar to the effect of treating whole cells with MMS, and consistent with hyper-activation of intra-mitochondrial PARP-1 (Du et al., 2003). Treatment of isolated mitochondria with FK866, with or without MMS, resulted in even larger decreases in mitochondrial NAD+ levels. Similar decreases in NAD+ were observed after treatment of mitochondria prepared by an alternative method (FIG. 13). These data indicated that mitochondria possess an NAD+ salvage pathway and that inhibition of Nampt in the organelle results in decreased NAD+.

As a further test, we determined whether the protection provided by Nampt required an intact mitochondrial NAD+ salvage pathway. During the course of these experiments, we were fortunate that an exclusively mitochondrial NAD+ biosynthetic enzyme, Nmnat-3, was discovered (Berger et al., 2005). We reasoned that if Nampt boosts cell survival by increasing the synthesis of NAD+ in mitochondria rather than in the cytoplasm or the nucleus, then knocking down Nmnat-3 should diminish Nampt-mediated protection against MMS. If not, then knocking down Nmnat-3 should have no effect on cell protection.

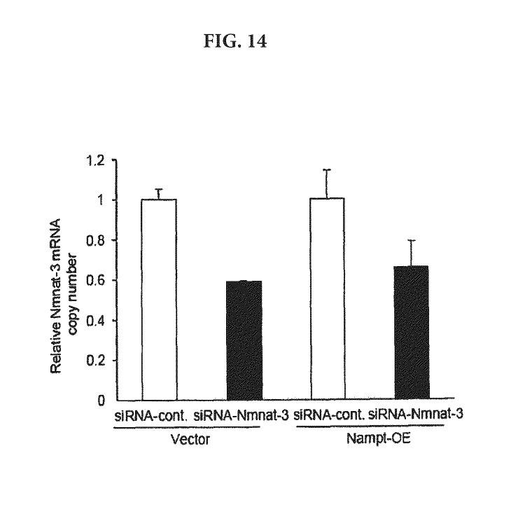

Nmnat-3 was knocked down ˜40% using an siRNA construct in HEK293 cells expressing a stably integrated Nampt construct (FIG. 14). Cells were then tested for resistance to MMS. As shown in FIG. 7H, knockdown of Nmnat-3 significantly reduced Nampt-mediated protection against MMS, demonstrating that a complete mitochondrial NAD+ salvage pathway is necessary for Nampt to provide resistance to MMS. No protection from MMS was seen by overexpressing Nmnat-3 (data not shown), which is in accordance with reports that overexpressing the cytoplasmic form of Nmnat (Nmnat-1) has no effect on NAD+ levels (Araki et al., 2004; Revollo et al., 2004).

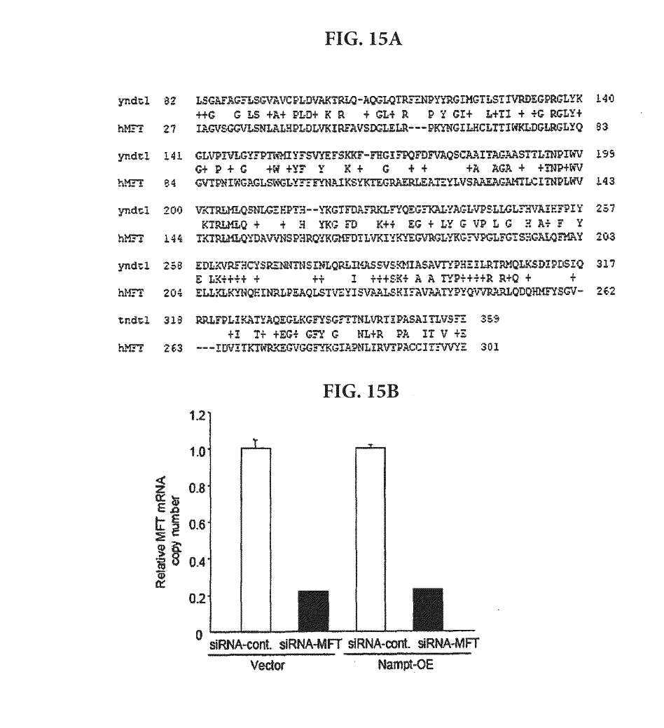

The alternative hypothesis that Nampt increases mitochondrial NAD+ by promoting NAD+ import from the cytoplasm seemed less likely given our observation that mitochondria retain higher NAD+ levels than the cytoplasm during MMS treatment. Nevertheless, we searched for evidence of a mammalian mitochondrial NAD+ transporter. Although mitochondrial NAD+ transport has not been described for mammals, two mitochondrial NAD+ transporters were recently discovered in S. cerevisiae (Todisco et al., 2006). We identified a putative human homolog called hMFT (identity=35% to yeast Ndt1; accession NM_030780; FIG. 15A). Knocking down of hMFT did not, however, affect the ability of Nampt to protect from MMS (FIGS. 15B and 7I). We cannot rule out the possibility that another NAD+ transporter exists and that mitochondria can import NAD+ from the cytoplasm but, taken together, we believe that the simplest explanation of the data is that Nampt is active in mitochondria.

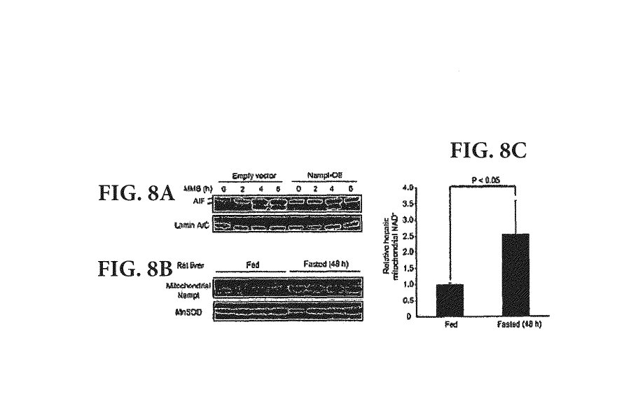

It is generally accepted that depletion of NAD+ stimulates a number of pro-apoptotic pathways, including the relocalization of AIF from the outer mitochondrial membrane to the nucleus (Di Lisa and Bernardi, 2006; Porcu and Chiarugi, 2005; Yu et al., 2002). As shown in FIG. 8A, Nampt overexpression suppressed translocation of AIF to the nucleus in response to MMS, demonstrating that Nampt lies upstream of this major apoptotic pathway.

### Fasting Increases Mitochondrial Nampt and NAD+ In Vivo

Finally, we tested whether these data were relevant to the in vivo situation. If our hypothesis that Nampt is a nutrient-responsive regulator of mitochondrial NAD+ we should observe increased levels of Nampt in the mitochondria of fasted rats, concomitant with increased levels of mitochondrial NAD+. To our knowledge, in vivo changes in mitochondrial NAD+ have not been examined previously. Rats were fasted 48 hours, their livers excised, and mitochondria were purified as described above. Mitochondrial fractions were divided for Western blotting and for NAD+ determinations using HPLC-MS. After the 48 hour fast there was a dramatic rise in Nampt levels in the mitochondrial fractions (FIG. 8B) and there was a concomitant increase in mitochondrial NAD+ levels in the mitochondrial extracts from the fasted animals (FIG. 8C). Thus, our results in cell culture extrapolate meaningfully to the in vivo situation.

### Discussion

The importance of mitochondrial function to the rate of progression of age-related diseases such as cancer, diabetes and neurodegeneration has become increasingly apparent in recent years (Lin et al., 2005; St-Pierre et al., 2006). Yet little is currently known about the intracellular concentration of NAD+ in mitochondria, whether it fluctuates in response to diet, or whether these changes influence key cellular functions such as apoptosis. In this study, we have accurately determined NAD+ concentrations in mammalian mitochondria, identified mitochondrial NAD+ as a determinant of cell survival, and shown that mitochondrial NAD+ levels are dramatically upregulated by nutrient restriction in vitro and in vivo. One of the more surprising findings of the study was the observation that mitochondria can maintain physiological levels of NAD+ during genotoxic stress and promote cell survival, even if NAD+ in the cytoplasm and nucleus has fallen well below normal physiological levels. We refer to the ability of mitochondria to dictate cell survival as the “Mitochondrial Oasis Effect.”

This study also shows that Nampt is a stress- and diet-responsive regulator of mitochondrial NAD+ in mammalian cells. The data strongly suggests that Nampt is both present and functional within mitochondria, directly upstream of the exclusively mitochondrial NAD+ biosynthetic enzyme Nmnat-3. Although we cannot and do not rule out other mechanisms by which mitochondria obtain NAD+, such as NAD+ import or via alternative NAD+ biosynthetic routes (Bieganowski and Brenner, 2004), the fact that Nampt activity is required to maintain NAD+ levels in isolated mitochondria is strong evidence that Nampt plays a functional role within these organelles. Given the central role of Nampt in NAD+ biosynthesis, it is likely that Nampt activity is not simply regulated at the gene expression level but at multiple levels, including by substrate availability and potentially by post-translational modification.

Given that numerous enzymes in mitochondria are limited by NAD+ availability, including the sirtuins SIRT3 and SIRT4, which are known to regulate GDH and AceCS2, respectively, it will be interesting to explore the potential impact of mitochondrial NAD+ levels on the metabolism and health of various organs. Perhaps diet-induced increases in mitochondrial NAD+ contribute to not only the increased resistance of calorie restricted rodents to toxins, but also the changes in fatty acid metabolism and respiration that occur with reduced caloric intake (Ando et al., 2002; Campisi, 2003; Higami and Shimokawa, 2000; Migliaccio et al., 1999; Zhang and Herman, 2002).

The events that lead to PARP-induced apoptosis remain poorly understood but it is known that AIF relocalization is a key event (Di Lisa and Bernardi, 2006; Yu et al., 2002). In this study, we find that overexpression of Nampt leads to an attenuation of AIF relocalization. Given that NAMPT lies upstream of AIF, it will be interesting to test whether SIRT3 or SIRT4 associate with and/or modify AIF or other determinants of apoptosis such as the permeability transition pore (PTP).

Our observation that Nampt is a stress- and nutrient-responsive gene that promotes cell survival via SIRT3 and SIRT4 lends further support to the hypothesis that NAMPT is a functional homolog of the yeast PNC1 longevity gene (Anderson et al., 2003; Bitterman et al., 2003). Transgenic mouse experiments are in progress to determine the effect of overexpressing NAMPT. We hypothesize that these animals might have increased resistance to cell stress, altered metabolism, and disease resistance (North and Sinclair, 2007).

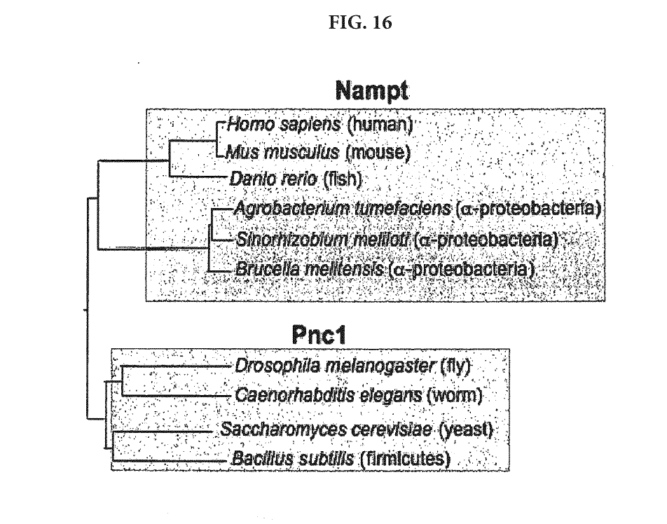

Because NAD+ is such an ancient molecule, insights into the biology of NAD+ can provide clues about the early evolution of life on earth (Brenner, 2005). There is evidence that cells have used NAD+ as a nutrient sensor that dictates survival for a very long time, possibly predating the evolution of eukaryotes. Homologs of Nampt and Sir2 are found in bacterial relatives of mitochondria (Smith et al., 2000) and increased NAD+ levels provide bacterial resistance to heat, salt stress, and glucose restriction, for reasons that are not yet clear (Foster et al., 1990). A phylogenetic comparison of NAM-metabolizing enzymes from various species shows that vertebrates utilize a pathway more closely related to the organisms that gave rise to the first mitochondria (Andersson et al., 2003) than to S. cerevisiae, C. elegans and D. melanogaster (FIG. 16). This indicates that NAD+ levels may have controlled cell survival in the bacteria that gave rise to mitochondria, and these survival pathways have been conserved up to the present day in mammals.

In summary, we have shown that mitochondrial NAD+ levels influence cell survival following genotoxic stress and that these levels are considerably higher after nutrient deprivation. We hope that these insights into the importance of mitochondrial NAD+ will facilitate a new understanding of and facilitate the development of novel approaches to treating diseases such as cancer and neurodegeneration.

### REFERENCES FOR EXAMPLE 2

Anderson, R. M., Bitterman, K. J., Wood, J. G., Medvedik, O., Cohen, H., Lin, S. S., Manchester, J. K., Gordon, J. I., and Sinclair, D. A. (2002). Manipulation of a Nuclear NAD⁺ Salvage Pathway Delays Aging without Altering Steady-state NAD+ Levels. J Biol Chem 277, 18881-18890.
Anderson, R. M., Bitterman, K. J., Wood, J. G., Medvedik, O., and Sinclair, D. A. (2003). Nicotinamide and Pnc1 govern lifespan extension by calorie restriction in S. cerevisiae. Nature 423, 181-185.
Andersson, S. G., Karlberg, O., Canback, B., and Kurland, C. G. (2003). On the origin of mitochondria: a genomics perspective. Philos Trans R Soc Lond B Biol Sci 358, 165-177; discussion 177-169.
Ando, K., Higami, Y., Tsuchiya, T., Kanematsu, T., and Shimokawa, I. (2002). Impact of aging and life-long calorie restriction on expression of apoptosis-related genes in male F344 rat liver. Microsc Res Tech 59, 293-300.
Araki, T., Sasaki, Y., and Milbrandt, J. (2004). Increased nuclear NAD biosynthesis and SIRT1 activation prevent axonal degeneration. Science 305, 1010-1013.
Barile, M., Cozzani, E., Anonide, A., Usiglio, D., Burroni, A., and Guarrera, M. (1996). Is contact allergy rare in psoriatics? Contact Dermatitis 35, 113-114.
Berger, F., Lau, C., Dahlmann, M., and Ziegler, M. (2005). Subcellular compartmentation and differential catalytic properties of the three human nicotinamide mononucleotide adenylyltransferase isoforms. J Biol Chem 280, 36334-36341.
Bieganowski, P., and Brenner, C. (2004). Discoveries of nicotinamide riboside as a nutrient and conserved NRK genes establish a Preiss-Handler independent route to NAD+ in fungi and humans. Cell 117, 495-502.
Bitterman, K. J., Medvedik, O., and Sinclair, D. A. (2003). Longevity regulation in Saccharomyces cerevisiae: linking metabolism, genome stability, and heterochromatin. Microbiol Mol Biol Rev 67, 376-399, table of contents.
Brenner, C. (2005). Evolution of NAD biosynthetic enzymes. Structure 13, 1239-1240.
Burkle, A. (2005). Poly(ADP-ribose). The most elaborate metabolite of NAD+. Febs J 272, 4576-4589.
Busch, H., Muramatsu, M., Adams, H., Steele, W. J., Liau, M. C., and Smetana, K. (1963). Isolation of Nucleoli. Exp Cell Res 24, SUPPL9:150-163.
Campisi, J. (2003). Cellular senescence and apoptosis: how cellular responses might influence aging phenotypes. Exp Gerontol 38, 5-11.
Cipriani, G., Rapizzi, E., Vannacci, A., Rizzuto, R., Moroni, F., and Chiarugi, A. (2005). Nuclear poly(ADP-ribose) polymerase-1 rapidly triggers mitochondrial dysfunction. J Biol Chem 280, 17227-17234.
Denu, J. M. (2005). The Sir2 family of protein deacetylases. Curr Opin Chem Biol 9, 431-440.
Di Lisa, F., and Bernardi, P. (2006). Mitochondria and ischemia-reperfusion injury of the heart: fixing a hole. Cardiovasc Res 70, 191-199.
Drevs, J., Loser, R., Rattel, B., and Esser, N. (2003). Antiangiogenic potency of FK866/K22.175, a new inhibitor of intracellular NAD biosynthesis, in murine renal cell carcinoma. Anticancer Res 23, 4853-4858.
Du, L., Zhang, X., Han, Y. Y., Burke, N. A., Kochanek, P. M., Watkins, S. C., Graham, S. H., Carcillo, J. A., Szabo, C., and Clark, R. S. (2003). Intra-mitochondrial poly(ADP-ribosylation) contributes to NAD+ depletion and cell death induced by oxidative stress. J Biol Chem 278, 18426-18433.
Foster, J. W., Park, Y. K., Penfound, T., Fenger, T., and Spector, M. P. (1990). Regulation of NAD metabolism in Salmonella typhimurium: molecular sequence analysis of the bifunctional nadR regulator and the nadA-pnuC operon. J Bacteriol 172, 4187-4196.
Frye, R. A. (2000). Phylogenetic classification of prokaryotic and eukaryotic Sir2-like proteins. Biochem Biophys Res Commun 273, 793-798.
Fukuhara, A., Matsuda, M., Nishizawa, M., Segawa, K., Tanaka, M., Kishimoto, K., Matsuki, Y., Murakami, M., Ichisaka, T., Murakami, H., et al. (2004). Visfatin: A Protein Secreted by Visceral Fat that Mimics the Effects of Insulin. Science.
Gallo, C. M., Smith, D. L., Jr., and Smith, J. S. (2004). Nicotinamide clearance by Pnc1 directly regulates Sir2-mediated silencing and longevity. Mol Cell Biol 24, 1301-1312.
Gray, M. W., Burger, G., and Lang, B. F. (1999). Mitochondrial evolution. Science 283, 1476-1481.
Guarente, L. (2005). Calorie restriction and SIR2 genes—towards a mechanism. Mech Ageing Dev 126, 923-928.
Guarente, L., and Picard, F. (2005). Calorie restriction—the SIR2 connection. Cell 120, 473-482.
Haigis, M. C., Mostoslavsky, R., Haigis, K. M., Fahie, K., Christodoulou, D. C., Murphy, A. J., Valenzuela, D. M., Yancopoulos, G. D., Karow, M., Blander, G., et al. (2006). SIRT4 inhibits glutamate dehydrogenase and opposes the effects of calorie restriction in pancreatic beta cells. Cell 126, 941-954.
Hallows, W. C., Lee, S., and Denu, J. M. (2006). Sirtuins deacetylate and activate mammalian acetyl-CoA synthetases. Proc Natl Acad Sci USA 103, 10230-10235.
Higami, Y., and Shimokawa, I. (2000). Apoptosis in the aging process. Cell Tissue Res 301, 125-132.
Horton, J. K., Stefanick, D. F., and Wilson, S. H. (2005). Involvement of poly(ADP-ribose) polymerase activity in regulating Chk1-dependent apoptotic cell death. DNA Repair (Amst) 4, 1111-1120.
Imai, S., Armstrong, C. M., Kaeberlein, M., and Guarente, L. (2000). Transcriptional silencing and longevity protein Sir2 is an NAD-dependent histone deacetylase. Nature 403, 795-800.
James, S. J., Muskhelishvili, L., Gaylor, D. W., Turturro, A., and Hart, R. (1998). Upregulation of apoptosis with dietary restriction: implications for carcinogenesis and aging. Environ Health Perspect 106 Suppl 1, 307-312.
Kaeberlein, M., McVey, M., and Guarente, L. (1999). The SIR2/3/4 complex and SIR2 alone promote longevity in Saccharomyces cerevisiae by two different mechanisms. Genes Dev 13, 2570-2580.
Kitani, T., Okuno, S., and Fujisawa, H. (2003). Growth phase-dependent changes in the subcellular localization of pre-B-cell colony-enhancing factor. FEBS Lett 544, 74-78.
Kun, E., Zimber, P. H., Chang, A. C., Puschendorf, B., and Grunicke, H. (1975). Macromolecular enzymatic product of NAD+ in liver mitochondria. Proc Natl Acad Sci USA 72, 1436-1440.
Lin, J., Handschin, C., and Spiegelman, B. M. (2005). Metabolic control through the PGC-1 family of transcription coactivators. Cell Metab 1, 361-370.
Lin, S. J., Defossez, P. A., and Guarente, L. (2000). Requirement of NAD and SIR2 for lifespan extension by calorie restriction in Saccharomyces cerevisiae. Science 289, 2126-2128.
Matsui, T., Li, L., del Monte, F., Fukui, Y., Franke, T. F., Hajjar, R. J., and Rosenzweig, A. (1999). Adenoviral gene transfer of activated phosphatidylinositol 3′-kinase and Akt inhibits apoptosis of hypoxic cardiomyocytes in vitro. Circulation 100, 2373-2379.
Migliaccio, E., Giorgio, M., Mele, S., Pelicci, G., Reboldi, P., Pandolfi, P. P., Lanfrancone, L., and Pelicci, P. G. (1999). The p66shc adaptor protein controls oxidative stress response and life span in mammals. Nature 402, 309-313.
Noack, H., Kunz, W. S., and Augustin, W. (1992). Evaluation of a procedure for the simultaneous determination of oxidized and reduced pyridine nucleotides and adenylates in organic phenol extracts from mitochondria. Anal Biochem 202, 162-165.
North, B. J., and Sinclair, D. A. (2007). Sirtuins: a conserved key unlocking AceCS activity. Trends Biochem Sci 32, 1-4.
Nukala, V. N., Singh, I. N., Davis, L. M., and Sullivan, P. G. (2006). Cryopreservation of brain mitochondria: a novel methodology for functional studies. J Neurosci Methods 152, 48-54.
Pillai, J. B., Isbatan, A., Imai, S., and Gupta, M. P. (2005). Poly(ADP-ribose) polymerase-1-dependent cardiac myocyte cell death during heart failure is mediated by NAD+ depletion and reduced Sir2alpha deacetylase activity. J Biol Chem 280, 43121-43130.
Porcu, M., and Chiarugi, A. (2005). The emerging therapeutic potential of sirtuin-interacting drugs: from cell death to lifespan extension. Trends Pharmacol Sci 26, 94-103.
Revollo, J. R., Grimm, A. A., and Imai, S. (2004). The NAD biosynthesis pathway mediated by nicotinamide phosphoribosyltransferase regulates Sir2 activity in mammalian cells. J Biol Chem 279, 50754-50763.
Rogina, B., and Helfand, S. L. (2004). Sir2 mediates longevity in the fly through a pathway related to calorie restriction. Proc Natl Acad Sci USA 101, 15998-16003.
Rongvaux, A., Shea, R. J., Mulks, M. H., Gigot, D., Urbain, J., Leo, O., and Andris, F.
(2002). Pre-B-cell colony-enhancing factor, whose expression is up-regulated in activated lymphocytes, is a nicotinamide phosphoribosyltransferase, a cytosolic enzyme involved in NAD biosynthesis. Eur J Immunol 32, 3225-3234.
Samal, B., Sun, Y., Stearns, G., Xie, C., Suggs, S., and McNiece, I. (1994). Cloning and characterization of the cDNA encoding a novel human pre-B-cell colony-enhancing factor. Mol Cell Biol 14, 1431-1437.
Sauve, A. A., Moir, R. D., Schramm, V. L., and Willis, I. M. (2005). Chemical activation of Sir2-dependent silencing by relief of nicotinamide inhibition. Mol Cell 17, 595-601.
Sauve, A. A., and Schramm, V. L. (2003). Sir2 regulation by nicotinamide results from switching between base exchange and deacetylation chemistry. Biochemistry 42, 9249-9256.
Schwer, B., North, B. J., Frye, R. A., Ott, M., and Verdin, E. (2002). The human silent information regulator (Sir)2 homologue hSIRT3 is a mitochondrial nicotinamide adenine dinucleotide-dependent deacetylase. J Cell Biol 158, 647-657.
Smith, J. S., Brachmann, C. B., Celic, I., Kenna, M. A., Muhammad, S., Starai, V. J., Avalos, J. L., Escalante-Semerena, J. C., Grubmeyer, C., Wolberger, C., and Boeke, J. D. (2000).
A phylogenetically conserved NAD+-dependent protein deacetylase activity in the Sir2 protein family. Proc Natl Acad Sci USA 97, 6658-6663.
Solomon, J. M., Pasupuleti, R., Xu, L., McDonagh, T., Curtis, R., DiStefano, P. S., and Huber, L. J. (2006). Inhibition of SIRT1 catalytic activity increases p53 acetylation but does not alter cell survival following DNA damage. Mol Cell Biol 26, 28-38.
St-Pierre, J., Drori, S., Uldry, M., Silvaggi, J. M., Rhee, J., Jager, S., Handschin, C., Zheng, K., Lin, J., Yang, W., et al. (2006). Suppression of reactive oxygen species and neurodegeneration by the PGC-1 transcriptional coactivators. Cell 127, 397-408.
Tanny, J. C., Dowd, G. J., Huang, J., Hilz, H., and Moazed, D. (1999). An enzymatic activity in the yeast Sir2 protein that is essential for gene silencing. Cell 99, 735-745.
Tissenbaum, H. A., and Guarente, L. (2001). Increased dosage of a sir-2 gene extends lifespan in Caenorhabditis elegans. Nature 410, 227-230.
Tobin, A., Djerdjour, B., Journet, E., Neuburger, M., and Douce, R. (1980). Effect of NAD on Malate Oxidation in Intact Plant Mitochondria. Plant Physiol 66, 225-229.
Todisco, S., Agrimi, G., Castegna, A., and Palmieri, F. (2006). Identification of the mitochondrial NAD+ transporter in Saccharomyces cerevisiae. J Biol Chem 281, 1524-1531.
van Roermund, C. W., Elgersma, Y., Singh, N., Wanders, R. J., and Tabak, H. F. (1995). The membrane of peroxisomes in Saccharomyces cerevisiae is impermeable to NAD(H) and acetyl-CoA under in vivo conditions. Embo J 14, 3480-3486.
van Wijk, S. J., and Hageman, G. J. (2005). Poly(ADP-ribose) polymerase-1 mediated caspase-independent cell death after ischemia/reperfusion. Free Radic Biol Med 39, 81-90.
Viswanathan, M., Kim, S. K., Berdichevsky, A., and Guarente, L. (2005). A role for SIR-2.1 regulation of ER stress response genes in determining C. elegans life span. Dev Cell 9, 605-615.
Wood, J. G., Rogina, B., Lavu, S., Howitz, K., Helfand, S. L., Tatar, M., and Sinclair, D. (2004). Sirtuin activators mimic caloric restriction and delay ageing in metazoans. Nature 430, 686-689.
Yamada, K., Hara, N., Shibata, T., Osago, H., and Tsuchiya, M. (2006). The simultaneous measurement of nicotinamide adenine dinucleotide and related compounds by liquid chromatography/electrospray ionization tandem mass spectrometry. Anal Biochem 352, 282-285.
Yang, H., Lavu, S., and Sinclair, D. A. (2006). Nampt/PBEF/Visfatin: a regulator of mammalian health and longevity? Exp Gerontol 41, 718-726.
Yu, S. W., Wang, H., Poitras, M. F., Coombs, C., Bowers, W. J., Federoff, H. J., Poirier, G. G., Dawson, T. M., and Dawson, V. L. (2002). Mediation of poly(ADP-ribose) polymerase-1-dependent cell death by apoptosis-inducing factor. Science 297, 259-263.
Zhang, Y., and Herman, B. (2002). Ageing and apoptosis. Mech Ageing Dev 123, 245-260.

### EQUIVALENTS

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Those skilled in the art will recognize, or be able to ascertain using no more than routine experimentation, many equivalents of the specific embodiments of the invention described herein. Such equivalents are intended to be encompassed by the following claims.

What is claimed is:

1. A method for increasing mitochondrial biogenesis, increasing mitochondrial metabolism, or boosting mitochondrial activity or endurance in a muscle cell, comprising:
contacting a muscle cell with an agent that increases a protein level of nicotinamide nucleotide adenylyltransferase 3 (NMNAT3) or an activity level of NMNAT3 in the muscle cell,
wherein the agent is administered to a subject in need thereof in an effective amount to increase mitochondrial biogenesis, increase mitochondrial metabolism, or boost mitochondrial activity or endurance in the muscle cell.

2.-28. (canceled)

29. The method of claim 1, wherein the NMNAT3 agent comprises a substrate of NMNAT3.

30. The method of claim 29, wherein the substrate of NMNAT3 comprises nicotinamide mononucleotide (NMN).

31. The method of claim 29, wherein the substrate of NMNAT3 comprises nicotinic acid mononucleotide (NaMN).

32. The method of claim 29, wherein the substrate of NMNAT3 comprises reduced nicotinamide mononucleotide (NMNH).

33. The method of claim 1, wherein the muscle cell is a skeletal muscle cell.

34. The method of claim 1, wherein the agent is targeted to or administered into a muscle of the subject.

35. A method for treating a subject in which muscle cells would benefit from calorie restriction or exercise, comprising:
administering to a subject in need thereof an agent that increases a protein or activity level of SIRT3 in a muscle cell,
wherein the agent is administered to a subject in need thereof in an effective amount to treat the subject, and
wherein the agent comprises a nicotinamide nucleotide adenylyltransferase 3 (NMNAT3) agent that increases a protein or activity level of NMNAT3 in the cell.

36. The method of claim 35, wherein the NMNAT3 agent comprises a substrate of NMNAT3.

37. The method of claim 36, wherein the substrate of NMNAT3 comprises nicotinamide mononucleotide (NMN).

38. The method of claim 36, wherein the substrate of NMNAT3 comprises nicotinic acid mononucleotide (NaMN).

39. The method of claim 36, wherein the substrate of NMNAT3 comprises reduced nicotinamide mononucleotide (NMNH).

40. The method of claim 35, wherein the agent is targeted to or administered into a muscle of the subject.

41. The method of claim 35, wherein the treatment is for a mitochondrial disease, a metabolic disorder, a neurologic disorder, a muscular disorder, a cardiovascular disease, excessive weight, or obesity.

42. The method of claim 35, wherein the treatment is for insulin resistance, diabetes, a diabetes-related condition or disorder, or metabolic syndrome.

43. A method for regulating skeletal muscle metabolism or skeletal muscle energy homeostasis in a subject, comprising:
administering to a subject in need thereof an agent that modulates a protein or activity level of SIRT3 in a muscle cell,
wherein the agent is administered to a subject in need thereof in an effective amount to regulate skeletal muscle metabolism or skeletal muscle energy homeostasis in the subject, and
wherein the agent comprises a nicotinamide nucleotide adenylyltransferase 3 (NMNAT3) agent that modulates a protein or activity level of NMNAT3 in the cell.

44. The method of claim 43, wherein the NMNAT3 agent comprises a substrate of NMNAT3.

45. The method of claim 43, wherein the substrate of NMNAT3 comprises nicotinamide mononucleotide (NMN).

46. The method of claim 43, wherein the substrate of NMNAT3 comprises nicotinic acid mononucleotide (NaMN).

47. The method of claim 43, wherein the substrate of NMNAT3 comprises reduced nicotinamide mononucleotide (NMNH).


SEQUENCE LISTING (25 sequences)

SEQ ID NO: 1 - DNA, 22 bp, artificial sequence
  synthetic oligonucleotide
atctcgcagataggctatcagc

SEQ ID NO: 2 - DNA, 22 bp, artificial sequence
  synthetic oligonucleotide
aaccacgtaaccttacccaagg

SEQ ID NO: 3 - DNA, 23 bp, artificial sequence
  synthetic oligonucleotide
ataaaccctcttgcagttgcatc

SEQ ID NO: 4 - protein, 15 aa, mus musculus
DLMQRERGKLDGQDR

SEQ ID NO: 5 - DNA, 20 bp, artificial sequence
  synthetic oligonucleotide
tctaatcgccatagccttcc

SEQ ID NO: 6 - DNA, 21 bp, artificial sequence
  synthetic oligonucleotide
ggttgttaaagggcgtattgg

SEQ ID NO: 7 - DNA, 20 bp, artificial sequence
  synthetic oligonucleotide
tgatggtgaggtctggaatg

SEQ ID NO: 8 - DNA, 22 bp, artificial sequence
  synthetic oligonucleotide
gcaggttcaaatgaatcttcag

SEQ ID NO: 9 - DNA, 21 bp, artificial sequence
  synthetic oligonucleotide
ggtgaagatctaagacattta

SEQ ID NO: 10 - DNA, 24 bp, artificial sequence
  synthetic oligonucleotide
aaatccgctcgacactgtcctgaa

SEQ ID NO: 11 - DNA, 24 bp, artificial sequence
  synthetic oligonucleotide
ttgggatcagcaactgggtcctta

SEQ ID NO: 12 - DNA, 24 bp, artificial sequence
  synthetic oligonucleotide
ttcctccctggagaagagctatga

SEQ ID NO: 13 - DNA, 24 bp, artificial sequence
  synthetic oligonucleotide
tactcctgcttgctgatccacatc

SEQ ID NO: 14 - DNA, 24 bp, artificial sequence
  synthetic oligonucleotide
acttcgtaggctggcctcaattct

SEQ ID NO: 15 - DNA, 24 bp, artificial sequence
  synthetic oligonucleotide
acatctggtttcagatggcctcca

SEQ ID NO: 16 - DNA, 24 bp, artificial sequence
  synthetic oligonucleotide
agagctccacgggaacatgtttgt

SEQ ID NO: 17 - DNA, 24 bp, artificial sequence
  synthetic oligonucleotide
atgtacccagcgtgatggacaggt

SEQ ID NO: 18 - DNA, 24 bp, artificial sequence
  synthetic oligonucleotide
tggccagagatcacctacaccaaa

SEQ ID NO: 19 - DNA, 24 bp, artificial sequence
  synthetic oligonucleotide
tgatgatgcctcagcaccttcact

SEQ ID NO: 20 - DNA, 24 bp, artificial sequence
  synthetic oligonucleotide
agggatttgttcctgggctgtttg

SEQ ID NO: 21 - DNA, 24 bp, artificial sequence
  synthetic oligonucleotide
aaatccaccgacgccttctttcct

SEQ ID NO: 22 - DNA, 24 bp, artificial sequence
  synthetic oligonucleotide
ttctacaatgagctgcgtgtggct

SEQ ID NO: 23 - DNA, 24 bp, artificial sequence
  synthetic oligonucleotide
ttaatgtcacgcacgatttcccgc

SEQ ID NO: 24 - protein, 279 aa, Sacharomyces cerevisiae
LSGAFAGFLSGVAVCPLDVAKTRLQAQGLQTRFENPYYRGIMGTLSTIVRDEGPRGLYKGLVPIVLGYFPTWMIYFSVYEFSKKFFHGIFPQFDFVAQSCAAITAGAASTTLTNPIWVVKTRLMLQSNLGEHPTHYKKGTFDAFRKLFYQEGFKALYAGLVPSLLGLFHVAIHFPIYEDLKVRFHCYSRENNTNSINLQRLIMASSVSKMIASAVTYPHEILRTRMQLKSDIPDSIQRRLFPLIKATYAQEGLKGFYSGFTTNLVRTIPASAITLVSFE

SEQ ID NO: 25 - protein, 275 aa, Homo sapiens
IAGVSGGVLSNLALHPLDLVKIRFAVSDGLELRPKYNGILHCLTTIWKLDGLRGLYQGVTPNIWGAGLSWGLYFFFYNAIKSYKTEGRAERLEATEYLVSAAEAGAMTLCITNPLWVTKTRLMLQYDAVVNSPHRQYKGMFDTLVKIYKYEGVRGLYKGFVPGLFGTSHGALQFMAYELLKLKYNQHINRLPEAQLSTVEYISVAALSKIFAVAATYPYQVVRARLQDQHMFYSGVIDVITKTWRKEGVGGFYKGIAPNLIRVTPACCITFVVYE